(19) United States
(12) Patent Application Publication
(10) Pub. No.: US 2025/0342972 A1
(43) Pub. Date: Nov. 6, 2025

(54) SYSTEM AND METHODS FOR GENERATING CLINICAL PREDICTIONS BASED ON MULTIMODAL MEDICAL DATA

(71) Applicant: SOPHiA GENETICS S.A., Rolle (CH)

(72) Inventors: Thierry COLIN, Bordeaux (FR); Loïc Cédric FERRER, Bordeaux (FR); Olivier GALLINATO, Bordeaux (FR); Guillaume Jean Lucien Etchepare, Gradignan (FR); Paul Frédéric Jules Bernard, Bordeaux (FR); Yves Keyne Le Moigne, Bordeaux (FR)

(21) Appl. No.: 19/195,635

(22) Filed: Apr. 30, 2025

Related U.S. Application Data
(60) Provisional application No. 63/641,412, filed on May 1, 2024.
(60) Provisional application No. 63/641,413, filed on May 1, 2024.
(60) Provisional application No. 63/676,166, filed on Jul. 26, 2024.

(51) Int. Cl. G16H 50/30; G06N 20/00; G16H 15/00

(52) U.S. Cl. CPC G16H 50/30; G06N 20/00; G16H 15/00

(57) ABSTRACT

Systems and methods for training predictive models for generating clinical predictions from multimodal medical data include receiving multimodal medical data of one or more medical subjects, and preprocessing and aggregating one or more features of the multimodal medical data. Further, for each cohort of medical subjects from the medical subjects, the method includes training one or more predictive models to generate a clinical prediction for each of the diseases based on the features of the multimodal medical data and deploying the predictive models to a model bank. The predictive models are used for making clinical predictions based on multimodal medical data of individual medical subjects. Use of multimodal medical data improves accuracy of the clinical predictions. Further, deploying predictive models on the model bank improves accessibility and useability of the predictive models.

### CROSS REFERENCE TO RELATED APPLICATIONS

The present application claims the benefit of U.S. Patent Application No. 63/676,166 for SYSTEM AND METHODS FOR GENERATING CLINICAL PREDICTIONS BASED ON MULTIMODAL MEDICAL DATA, filed Jul. 26, 2024; U.S. Patent Application No. 63/641,413 for AN IMPROVED MULTIMODAL PIPELINE FOR TREATMENT EFFICACY ANALYSIS filed May 1, 2024; and U.S. Patent Application No. 63/641,412 for A METHOD TO PREDICT INDIVIDUAL TREATMENT RESPONSES BASED ON MULTIMODAL DATA filed May 1, 2024, the entire contents of which are incorporated herein by reference.

### TECHNICAL FIELD

The present disclosure relates to medical data processing in general, and more specifically to systems and methods for generating clinical predictions based on multimodal medical data.

### BACKGROUND

Many diseases, such as cancer, are inherently multifaceted. Several patients suffering from the same disease can expect different outcomes and responses to the same treatments. Symptoms and rate of progression of the diseases may also differ from patient to patient. The diseases can have different genomic causes, such as mutations in distinct genes that disrupt unrelated pathways leading to the proliferation of cells and the development of tumors within a given tissue. Since the genetic make-up of each individual is different, the mutations may affect each individual differently. Further, as efficacy of some treatments depends on the affected pathway, the genomic causes of a disease can strongly influence the likely treatment response. The specific stage of such disease, the size and number of tumors, and detailed phenotypic expression of the disease are, in many cases, good predictors of the likely evolution/progression of the disease and its response to different treatment options. Furthermore, the condition of the affected patient unrelated to the disease, including their general health, history, metabolism, lifestyle, trauma/injuries, and genomic make-up, can strongly impact the expected prognosis and tolerance to different treatment options. It is therefore crucial to tailor the prognosis and the evaluation of treatment options to each affected subject.

Because of the multifaceted nature of such diseases, personalized predictions can rely on a combination of data from different modalities, which can utilize the genotypical or phenotypical information of the patients. Analyses of the genotype can focus on specific genes, in an effort to identify the causal mutations. Alternatively, markers assessing the general state of the genome, such as the tumor mutational burden or genome instability indexes, can be pivotal in predicting the response to potential treatments. The phenotype can be assessed at multiple levels, ranging from analyses of transcriptomes, proteomes, and metabolomes to imaging of cells or tissues, and data collected at the patient level, including their clinical history and demographic data, for example. Since the contribution of these different data types for making clinical predictions is a priori unknown, in most cases, there is a need for tools that consider data from multiple modalities jointly in clinical settings.

Analyses of multiple types of data, also known as multimodal analyses, are complicated by the inherently different formats of distinct datasets. For instance, radiological data typically consist of one or a series of images covering various areas of different tissues. Genomic data, on the other hand, primarily includes sequencing reads, which must be analyzed using bioinformatic tools before either extracting the identity of individual mutations, or assessing the genome state. Other types of data can come in a further variety of formats, and all data types can be collected at the same or different time points. Existing solutions struggle with reconciling data received in multiple modalities, leading to a potential mismatch between the number of collection points or the collection times across data types. Further, the data associated with certain patients may not necessarily be complete, thereby requiring other means/tools to impute the missing values in a manner that does not substantially affect the accuracy of the predictions. In many instances, it is also important to identify features/factors that significantly contribute to at least one of the likelihoods of onset of diseases, progression of diseases, potential treatment outcomes for different kinds of diseases, and the like. Additionally, such features/factors may also differ for different groups of individuals having similar genetic or biological characteristics.

Solutions and methods exist utilizing machine learning models to make clinical predictions, whether assessing unimodal or multimodal data (e.g., European Patent Application No. EP4287212A1 for MACHINE LEARNING PREDICTIVE MODELS OF TREATMENT RESPONSE). The training of models requires carefully curated datasets, where in-house scripts or manual intervention may be required to evaluate, classify, and organize heterogenous data in a usable database. In addition, many raw data types need pre-processing before being used to train machine learning models. For example, in the case of some deep learning radiomics models, features may be extracted from radiomic images (e.g., European Patent Application No. EP 4287142A1 for DEEP LEARNING MODELS OF RADIOMICS FEATURES EXTRACTION). Similarly, identifying variants or genomic features from sequence data requires bioinformatic pipelines. Solutions exist for some of these steps, but such solutions exist as separate tools, preventing the routine adoption of multimodal clinical predictions in healthcare technologies.

Therefore, there is a need for a system capable of ingesting and reconciling different modalities of data associated with patients, for developing and deploying models that generate clinically useful insights using the multimodal medical data.

The response to treatments can vary among diseased individuals, for example as a function of the specifics of the disease, the overall condition of the individual, and/or their genomic make up. Administering an ineffective treatment can not only be costly but can also harm the individual without offering any benefit. It is therefore crucial to provide healthcare professionals with a means to identify, out of different diseased individuals, those most likely to benefit from a given treatment.

Having an improved pipeline for the analysis of treatment efficacy is paramount in healthcare, clinical, pharmaceutical, and research settings. Central to any analysis pipeline is the development and refinement of the underlying predictive models that accurately assess outcomes. Moreover, intelligently weighted factors within these models play a crucial role in enhancing the model's predictive capabilities and accuracy. By assigning appropriate weights to various input parameters based on their relative importance and relevance, these factors ensure that the model captures the nuanced interplay of diverse variables influencing treatment outcomes. Intelligently weighted factors enable the model to discern subtle relationships and prioritize influential predictors, thereby improving the accuracy and robustness of predictions. Thus, many traditional models that do not contemplate dynamic pipelines or intelligent preprocessing, fail to accurately and effectively deliver outcome results.

In essence, an accurate yet easily deployable in routine pipeline for treatment efficacy analysis hinges on the synergy between perfected models, intelligently weighted factors, factor amalgamation, and strategic division between training and testing sets. By harnessing the power of advanced analytics and data-driven insights, an improved pipeline would manifest improvement in clinical practice, research, and healthcare.

The effect of certain treatments can be evaluated through clinical trials, where a selection of the diseased individuals receives a treatment and the other half serve as a control or, using real-world data, where a portion of the observed individuals have received a given treatment. The health outcome can then be compared between those individuals that received the treatment and those that did not. Variation among individuals in the response to the treatment can however blur the effect of the treatment when performing comparisons among heterogenous groups. It is therefore important to consider intra-group variation in the treatment response and develop tools to extract individual predictions from such datasets.

The treatment effect may therefore differ from one patient to another, referring to a heterogeneous treatment effect. The observed response to treatment can be modeled for each individual based on features measured before treatment initiation. Such models can then help predict whether a new patient, for whom features are available, is likely to respond to the treatment. However, each individual either belongs to the treatment or the control arms, so that the response to treatment (difference between control and treatment) cannot be measured directly at the individual level. Applying a potential outcome framework can help tackle this challenge. It is indeed possible to model the outcome (e.g., as survival) of individuals from the control group based on their measured features. An equivalent model can be generated for the treatment group, and the two models can be used to predict the outcome for each individual if they had received the treatment and if they had been part of the control group. The treatment effect can then be computed by comparing these two predictions, for example using the difference or ratio.

Any modelling effort inserts uncertainty and the predictions come with confidence levels. If the predictions are done independently for the control and treatment scenarios, two different sources of uncertainty result. When combined to obtain the predicted treatment benefits, the resulting uncertainty becomes large, inflating the confidence intervals and decreasing the accuracy of the model.

As a result, there is a need to provide a statistical approach to predict with more accuracy, at the individual level, the benefit of receiving a given treatment compared to a reference based on data for individuals that have either received a given treatment or not received the treatment. To ease application in routine practice, this statistical approach may preferably be implementable into a system that can digest and organize multimodal data, impute missing data, and train and deliver models.

### SUMMARY

This Summary is provided to introduce a selection of concepts in a simplified form that are further described below in the Detailed Description. This Summary is not intended to identify key features or essential features, nor is it intended to limit the scope of the claims included herewith.

In an aspect, embodiments of the present disclosure are directed to a method for training predictive models to generate clinical predictions from multimodal medical data. The method includes receiving, by a processor, multimodal medical data of one or more medical subjects. The method also includes preprocessing and aggregating, by the processor, one or more features of the multimodal medical data. Further, the method includes training, by the processor, one or more predictive models to generate a clinical prediction based on the one or more features of the multimodal medical data for each cohort of medical subjects from the one or more medical subjects. The method includes deploying, by the processor, the one or more predictive models to a model bank.

In some embodiments, the multimodal medical data may include any one or a combination of, genomic data, clinical data, radiological data, and biological data.

In some embodiments, for preprocessing the one or more features of the multimodal medical data, the method may include any one or a combination of, when the multimodal medical data may be received in the form of a plurality of unimodal medical data, reconciling the plurality of unimodal medical data to obtain the multimodal medical data of the one or more medical subjects, imputing missing values in the multimodal medical data, cleaning the multimodal medical data for errors, and performing feature extraction on the multimodal medical data.

In some embodiments, the method may include determining a subset of features from the multimodal medical data that are statistically associated with the clinical prediction. The identification of the features to be included in the multimodal predictive models may involve ranking the features based on their univariate association with the clinical outcome, using parameters and filtering criteria that are optimized during the model training.

In some embodiments, the performance of the one or more predictive models may be tested using nested cross-validation.

In some embodiments, the one or more medical subjects are grouped into one or more of the cohorts of medical subjects based on at least one feature in the multimodal medical data, such as their treatment history.

In some embodiments, the one or more predictive models may be trained for generating the clinical prediction for each treatment option associated with one or more diseases.

In some embodiments, the one or more predictive models may be trained for generating the prediction for the effect of a treatment associated with one or more diseases.

In some embodiments, the one or more predictive models may be trained for identifying subsets of patients that are predicted to respond differently to a given treatment option.

In some embodiments, the contribution of each feature to the clinical prediction is evaluated independently. The method includes, for each feature included in the multimodal dataset, randomly shuffling values associated with the feature of medical subjects in the cohort of medical subjects to generate at least one pseudo-replicate dataset, testing performance of the one or more predictive models on the pseudo-replicate dataset, and quantifying the contribution of the feature based on the decrease of model performance resulting from the shuffling of the feature values.

In another aspect, embodiments of the present disclosure are directed to a system having a processor, and a memory coupled to the processor. The memory includes processor-executable instructions, which, on execution, cause the processor to implement the method for training predictive models to generate clinical predictions from multimodal medical data. In a further aspect, embodiments of the present disclosure are directed to a non-transitory computer-readable medium having instructions to implement the method for training predictive models to generate clinical predictions from multimodal medical data.

In an additional aspect, embodiments of the present disclosure are directed to a system for generating clinical predictions based on multimodal medical data. The system includes a processor, and a memory coupled to the processor. The memory includes processor-executable instructions, which, on execution, cause the processor to receive multimodal medical data associated with a medical subject. The processor is further caused to generate at least one clinical prediction based on the multimodal medical data using a corresponding predictive model for each available treatment option for a disease. The processor is further configured to transmit the at least one clinical prediction for each available treatment option to a computing device of the medical subject, a third party, a clinician, or other relevant individual.

In some embodiments, the processor may be further configured to preprocess the multimodal medical data.

In some embodiments, the processor may be further configured to determine contribution of one or more features in the multimodal medical data by, for each feature from the one or more features, randomly varying a value associated with the feature in the multimodal medical data to obtain a corresponding pseudo-replicate data, generating at least one pseudo-replicate prediction based on the pseudo-replicate data using the corresponding predictive model, and determining the contribution of the feature based on a difference between the at least one pseudo-replicate prediction and the at least one clinical prediction.

In some embodiments, the processor may be further configured to determine contribution of one or more features in the multimodal medical data by, for each feature from the one or more features, randomly shuffling among subjects the values associated with the feature in the multimodal medical data, while keeping the values of the other features as set up based on the received data, to obtain a corresponding pseudo-replicate data, generating at least one pseudo-replicate prediction based on the pseudo-replicate data using the corresponding predictive model, and determining the contribution of the feature based on an accuracy difference between the at least one pseudo-replicate prediction and the at least one clinical prediction.

In some embodiments, to generate the at least one clinical prediction, the processor may be configured to retrieve the corresponding predictive model from a model bank, based on the multimodal medical data and the treatment option available for the disease.

The disclosure may provide a system for generating clinical predictions from multimodal medical data, comprising:
a. a processor configured to ingest data belonging to different modalities and available in various formats;
b. a data management engine configured to reconcile the different types of data, creating a multilevel multimodal database wherein each datapoint, independent of the modality, is associated with a specific subject;
c. a feature extraction module configured to process the data and extract features of interest;
d. a data aggregation module configured to aggregate data from one or more modalities, produce a list of features associated with subjects, and impute missing data to obtain values or distributions of values for all features in each subject;
e. a model development engine configured to develop clinical prediction models based on one or more groups of subjects, and to optimize and assess model performance using a validation technique;
f. a model bank for storing trained models, containing trained models adapted to make predictions for new subjects based on user goals and inputting subject features; and
g. an interface for reporting individual-level predictions and feature contributions.

In an embodiment, the data comprises distinct files per subject or multiple data types within the same file, wherein the data includes any of imaging data, genomic data, clinical data, and biological data.

In various embodiments, the imaging data comprises one or more of X-rays, MRI, PET scans, and CT scans, the genomic data comprises one or more of sequencing reads, genetic variants, gene expression profiles, genomic profiles, and methylation profiles, the clinical data comprises one or more of age, history, and health indicators, wherein the clinical data may be collected in time series from a given starting point, and/or the biological data comprises one or more of metabolomics, proteomics, pathology data, and results from blood or urine analyses.

In an embodiment, the feature extraction module utilizes tools to process images by automatically segmenting and extracting features comprising one or more of shape, intensity, texture, and the like.

In an embodiment, the feature extraction module utilizes tools to process sequencing data to, one or more of, identify genetic variants, assess genomic profiles, establish gene expression patterns, and extract other genomic features.

In an embodiment, the feature extraction module utilizes tools to extract features from metabolomic or proteomic data.

In an embodiment, the instant disclosure provides a computer-implemented method to predict the effect of a treatment to a condition, comprising:
a. receiving multimodal data for at least two cohorts of subjects having received different treatments;
b. developing a model for a clinical outcome independently for each of the at least two cohorts of subjects;
c. calculating a treatment benefit based on the clinical outcomes predicted with each of the models developed for the at least two cohorts of subjects; and
d. optimizing the models based on the predicted treatment benefit.

In an embodiment, the multimodal data is selected from a group consisting of clinical data, biological data, genomic data, and radiomic data.

In an embodiment, the received data is pre-processed prior to training the models.

The pre-processing may comprise one or more of any of quality checks and data cleaning, data imputation, data normalization, image processing, and analyses of genomic data.

In an embodiment, the different features are selected for the models for each of the at least two different cohorts of subjects.

In an aspect, the feature selection is integral to the step of optimizing the models.

The step of calculating the treatment benefit may further comprise comparing the clinical outcome predicted for each subject based on the model developed on the cohort having received a treatment and the clinical outcome predicted for each subject based on the model developed on the cohort not having received the treatment.

In an embodiment, the comparison comprises computing the difference between the clinical outcome predicted with the two models.

The comparison may further comprise the steps of:
a. defining a proportion c of individuals benefitting the most of the treatment; and
b. calculating the treatment benefit AD_{(c)} as the added average benefit observed in the top-ranked fraction c of the individuals compared to the average of the cohort.

The comparison may further comprise the steps of:
a. calculating AD_{(c)} for varying values of c; and
b. calculating the treatment benefit AD_{abc} as the integer of AD_{(c)} across the range of c values tested.

The comparison may further comprise the steps of:
a. calculating the correlation coefficient ρ between AD_{(c)} and c; and
b. calculating the treatment benefit AD_{wabc} by multiplying AD_{abc} by the absolute value of ρ.

The instant disclosure contemplates a system wherein the computer-implemented method described herein and any steps thereof may be integrated into said system.

Other aspects of the disclosure will be apparent from the following description and the appended claims.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIGS. 1A and 1B illustrate example architectures implementing a system for ingesting multimodal medical data for making clinical predictions, according to embodiments of the present disclosure.

FIG. 2 illustrates an example block diagram of the system, according to embodiments of the present disclosure.

FIG. 3 illustrates a flow diagram of training predictive models using multimodal medical data, according to embodiments of the present disclosure.

FIG. 4 illustrates a flowchart of an example method for generating clinical predictions based on multimodal medical data, according to embodiments of the present disclosure.

FIG. 5 illustrates an example computer system in which the embodiments of the system may be implemented, according to embodiments of the present disclosure.

FIG. 6 illustrates a graph of the progression of a health indicator over time for treatment and control groups.

FIG. 7 illustrates a flow chart diagram of the method for optimizing treatment effect predictions using multimodal data.

FIG. 8 is a flowchart below demonstrates a method of treatment outcome prediction as contemplated by the instant disclosure.

FIG. 9 illustrates a flow chart diagram of the process of training and testing predictive models using multimodal data for treatment outcome prediction.

FIG. 10 demonstrates a method facilitating an improved multimodal pipeline for treatment outcome prediction as contemplated by the instant disclosure.

FIG. 11 illustrates a schematic diagram of the process of model training and testing.

FIG. 12 illustrates a user interface for inputting patient data related to kidney cancer, featuring a model selection menu and a form for entering clinical parameters.

FIG. 13 shows a results interface displaying a patient-specific prediction for kidney cancer survival alongside a graph of feature contributions.

FIG. 14 depicts a prediction interface for assessing the risk of pT3a upstage after nephrectomy, with input fields for various clinical parameters.

### DETAILED DESCRIPTION

The particulars shown herein are by way of example and for purposes of illustrative discussion of the various embodiments only and are presented in the cause of providing what is believed to be the most useful and readily understood description of the principles and conceptual aspects of the methods and compositions described herein. In this regard, no attempt is made to show more detail than is necessary for a fundamental understanding, the description making apparent to those skilled in the art how the several forms may be embodied in practice.

The present disclosure will now be described by reference to more detailed embodiments. This present disclosure, however, may be embodied in different forms and should not be construed as limited to the embodiments set forth herein. Rather, these embodiments are provided so that this disclosure will be thorough and complete, and will fully convey the scope to those skilled in the art.

Unless otherwise defined, all technical and scientific terms used herein have the same meaning as commonly understood by those skilled in the art to which the present disclosure belongs. The terminology used in the description herein is for describing particular embodiments only and is not intended to be limiting. As used in the description and the appended claims, the singular forms ‘a,’ ‘an,’ and ‘the’ are intended to include the plural forms as well, unless the context clearly indicates otherwise.

Unless indicated to the contrary, the numerical parameters set forth in the following specification and attached claims are approximations that may vary depending upon the desired properties sought to be obtained and thus may be modified by the term about. At the very least, and not as an attempt to limit the application of the doctrine of equivalents to the scope of the claims, each numerical parameter should be construed in light of the number of significant digits and ordinary rounding approaches.

Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

### Definitions

Throughout the specification, the term “medical subject(s)” means and includes humans or animals undergoing clinical trials, clinical assessments, diagnoses, treatments, procedures, or the like, but not limited thereto.

Throughout the specification, terms “cohort” or “cohort of medical subjects” mean and include a set or a group of medical subjects satisfying a list of genetic, biological, medical, and/or clinical criteria.

The present disclosure solves the need for a multimodal “factory” system that is configured to ingest, process, and combine multimodal medical data, train predictive models to generate clinical predictions, and deploy the predictive models on a platform accessible by a plurality of stakeholders, such as including, but not limited to, healthcare practitioners, healthcare administrators, researchers conducting clinical trials, patients/medical subjects, and the like. The predictive models may be used for generating subject-level clinical predictions for individual medical subjects. By automating different processes involved in ingesting, processing, and managing multimodal medical data, the multimodal factory system provides a gateway for a wide community of health practitioners to personalize prognosis and treatment options for medical subjects. In one aspect, the “factory” element of the multimodal factory system refers to the system's ability to both train models and execute said models to determine treatment outcome efficacy, while permitting integration of such a system in third party systems. In such a sense, the “factory” element of the instant system enables a compact, comprehensive, and interoperable system.

### Multimodal “Factory” System

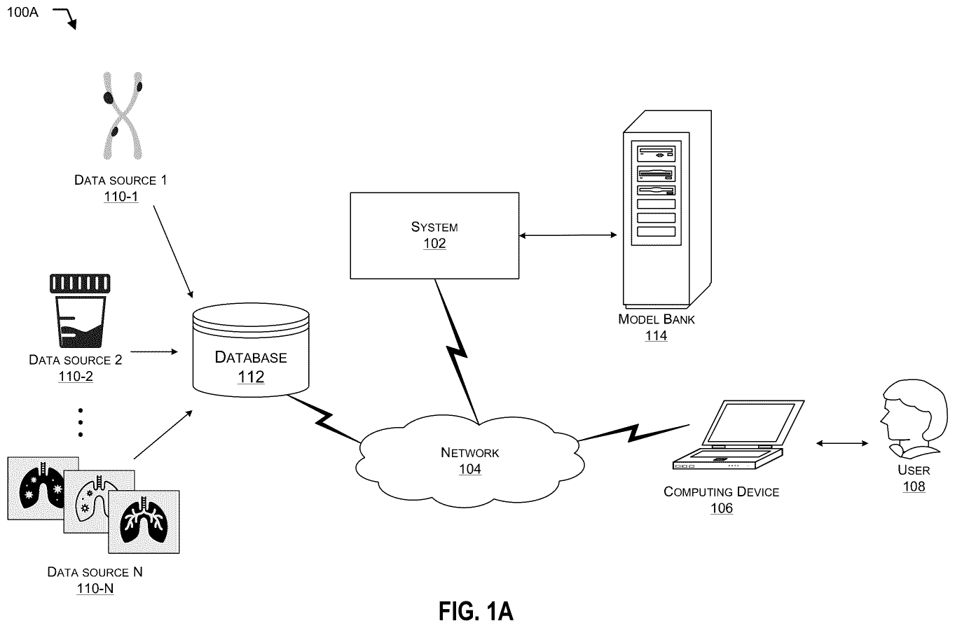

Referring to representations of architectures 100A and 100B shown in FIGS. 1A and 1B respectively, a multimodal factory system 102 (hereafter referred to as system 102) may be configured to train a set of predictive models 113 using multimodal medical data received from a plurality of data sources (such as data sources 110-1 to 110-N, collectively referred to as data sources 110), and store the predictive models 113 in a model bank 114. The data sources 110 may include multimodal medical data associated with individuals included in one or more cohorts of medical subjects 116, and/or individual medical subjects 118. Further, the system 102 may be configured to receive and process medical data associated with a medical subject 118 received from users (such as user 108) through a corresponding computing device (such as computing device 106) for inference. The system 102 may be configured to generate clinical predictions for the multimodal medical data of specific/individual medical subjects 118 received for inference.

As shown, the system 102 may be configured to receive multimodal medical data. The multimodal medical data may be a combination of including, but not limited to, genomic data, radiological data, clinical data, biological data, temporal data, and the like. Genomic data may include digital representation of genomic information, such as gene sequences, sequencing reads, genetic variants, gene expression profiles, genomic profiles, and methylation profiles, associated with the medical subjects. Genomic data may be obtained using a next generation sequencing (NGS) bioinformatics workflow, among other techniques. The genomic data may be in any one or combination of FASTQ file format, Binary Alignment Map (BAM) or Sequence Alignment Map (SAM) file format, and/or Variant Call Format (VCF) file format, based on the information being conveyed. In some embodiments, the system 102 may recognize the file format being received. For example, in one embodiment, the system 102 may be operative to automatically recognize the file format being received. In other embodiments, the system 102 may receive an input corresponding to the file format being received.

The term “variant” or “genomic variant” refers to a difference in a genomic sequence relative to a designated reference sequence. In bioinformatics data processing, a variant is uniquely identified based on its chromosomal position (chr, pos) and the deviation from the reference genome at that position (ref, alt). Variants may encompass single nucleotide variants (SNVs), known as single nucleotide polymorphisms (SNPs) when referring to populations, insertions or deletions (INDELs), copy number variants (CNVs), and structural genomic modifications such as large-scale rearrangements, duplications, translocations, etc.

Within a bioinformatics secondary analysis workflow, a variant caller may perform variant calling to generate one or more variant calls, which are typically documented in a Variant Call Format (VCF) file.

A “germline variant” refers to a variant inherited from at least one parent that differs from the wild-type genomic sequence as recorded in a reference database and is present in the majority of normal cells of an individual.

A “somatic variant,” also referred to as a “somatic mutation” or “somatic alteration,” denotes a genomic alteration arising in one or more somatic cells of an individual, such as those found in a tumor.

The term “mutation” or “mutated gene” refers to a gene in which at least one variant has been identified. A “mutated gene status” may be classified as “mutated” in such instances. Otherwise, said status may be denoted as “normal.” Such a classification is commonly utilized as a biomarker in cancer diagnostics and prognostics. For example, mutations in the ALK or EGFR genes have been established as particularly relevant in the context of lung cancer.

The term “mutational load,” “mutation load,” “mutation burden,” or “mutational burden,” and in the context of a tumor, “tumor mutational burden” (TMB) or “tumor mutational load,” refers to biomarkers that quantify the level of mutations, for example as the number of somatic mutations per megabase, within a given genomic sequence.

The term “Microsatellite Instability (MSI) status” or “MSI status” may refer to a genomic status characterized by an elevated level of insertions and/or deletions of nucleotides within microsatellite repeat regions, which may consist of mononucleotide repeats (homopolymers) or a number of nucleotide repeats (heteropolymers). Such a status may arise from a deficiency in the DNA mismatch repair system and may serve as a biomarker in cancer diagnostics and prognostics, particularly in uterine, colorectal, and gastric cancers, specifically Uterine Corpus Endometrial Carcinoma (UCES), Colon Adenocarcinoma (COAD), and/or Stomach Adenocarcinoma (STAD). A patient's MSI status is typically classified into one of the following categories: (1) Microsatellite Stable (MSS) (i.e., No detectable instability at any of the analyzed biomarker loci); (2) Microsatellite Instability-Low (MSI-L) (e.g., Instability detected in a single biomarker locus or instability detected with moderate confidence from an overall metric); and (3) Microsatellite Instability-High (MSI-H) (e.g., Instability detected in at least two biomarker loci or instability detected with high confidence from an overall metric).

The term “homologous recombination deficiency status” or “HRD status” refers to a genomic state that is frequently associated with deficiencies in the homologous recombination DNA repair pathway. HRD status may be categorized as (1) HRD-positive (HRD+), indicating a genomic state associated with a likely deficiency in the homologous recombination pathway; (2) HRD-negative (HRD−), indicating a genomic state associated with the likely absence of such a deficiency; or (3) HRD-uncertain/HRD-unknown, which indicates that the status cannot be determined based on available data.

The terms “genomic pathway” or “genetic pathway” refer to a defined set of genomic loci or the transcripts derived from them that encode for proteins with function in a shared biochemical or metabolic cascade. Such set of loci can be frequently associated with a particular biological or pathological condition.

Genomic data pertaining to a cohort of medical subjects 116 or a medical subject 118 may include, but is not limited to, the mutational status of the subject's 118 disease site, as determined through VCF files. The disease site may include tumor tissue or genetic material released from the tumor that is detected in circulating blood.

In one embodiment, genomic data for a cohort of medical subjects 116 or a medical subject 118 diagnosed with cancer, may include any given gene mutational status, such as one of EGFR and ALK mutational status. Tumor mutational status may be obtained using established methodologies, including but not limited to VCF files (derived from locally available NGS panels), Sanger sequencing, immunohistochemistry, and related analytical techniques.

In another embodiment, genomic data for a cohort of medical subjects 116 or a medical subject 118 diagnosed with cancer, may include information regarding tumor-specific mutations, including but not limited to mutations in at least one of: (1) EGFR; (2) ALK; (3) KRAS; (4) STK11/LKB1; (5) KEAP1; (6) PTEN; (7) PIK3CA; (8) TP53; (9) ROS1; (10) BRAF; and (11) NTRK1/2/3, in addition to components of the DNA repair pathway such as mismatch repair genes, POLE, BRCA2, and components of the interferon-gamma (IFN-γ) signaling pathway, including loss-of-function mutations in JAK1, JAK2, and beta-2-microglobulin (B2M).

In yet a further embodiment, genomic data for a cohort of medical subjects 116 or a medical subject 118 diagnosed with cancer, may include data on tumor immunogenicity indicators of other genomic indicators, such as TMB, MSI, HRD status, and defective mismatch repair (dMMR) status.

It is understood that genomic data collection may be performed initially to assess a baseline, or at multiple time points throughout disease progression and treatment.

Radiological data of the cohort of medical subjects 116 or the medical subjects 118 may include images collected from computerized tomography (CT), positron emission tomography (PET), PET/CT, magnetic resonance imaging (MRI), single-photon emission computerized tomography (SPECT), and the like, but not limited thereto. Radiological data may be stored as images, in formats compatible with those prescribed in Picture Archiving and Communication System (PACS).

The terms “medical image data,” “radiological data,” or “imaging data” refer to digital imaging data, including one or more images obtained for a patient at any point during the diagnostic and treatment process.

Radiological data pertaining to the cohort of medical subjects 116 or medical subjects 118, particularly those with cancer, may encompass at least one of the following:
Pre-baseline imaging, where available (e.g., millimetric injected CT scans of the cancer site, with a slice thickness <5 mm);
Baseline imaging, (e.g., millimetric injected CT scans of the cancer site, with a slice thickness <5 mm; as well as PET/CT, CT, and MRI scans, where available);
First and subsequent evaluation imaging (e.g., millimetric injected CT scans of the cancer site, with a slice thickness <5 mm; in addition to CT and MRI scans where available);
Imaging obtained during follow-up visits post-evaluation;
Imaging at disease progression, (e.g., millimetric injected CT scans of the cancer site, with a slice thickness <5 mm; and CT and MRI scans where available);
Quantification of metastases for each metastatic site at both baseline and first/further evaluations; and.

Evaluation criteria based on the Response Evaluation Criteria in Solid Tumors (RECIST), where applicable.

For example, radiological data for a cohort of medical subjects 116 or a medical subject 118 diagnosed with cancer, may include imaging-based assessments of clinical tumor burden. In such an example, radiological data may further comprise:
Pre-baseline imaging, where available (e.g., millimetric injected CT scans of the thorax, abdomen, and pelvis, with a slice thickness <5 mm);
Baseline imaging (e.g., millimetric injected CT scans of the thorax, abdomen, and pelvis, with a slice thickness <5 mm; in addition to PET/CT, brain CT, and brain MRI scans, if available);
First and subsequent evaluation imaging (e.g., millimetric injected CT scans of the thorax, abdomen, and pelvis, with a slice thickness <5 mm; and brain CT and MRI scans, where available);
Follow-up imaging post-evaluation;
Imaging obtained at progression, (e.g., millimetric injected CT scans of the thorax, abdomen, and pelvis, with a slice thickness <5 mm; and brain CT and MRI scans, where available);
Quantification of metastases for each metastatic site at both baseline and first/further evaluations; and
Evaluation criteria based on RECIST, if available.

In another embodiment, radiological data for a cohort of medical subjects 116 or medical subjects 118 diagnosed with Stage IV NSCLC may also include, but is not limited to:
Pre-baseline chest CT scans and scan dates;
Baseline CT scans of the thorax, abdomen, and pelvis (CT-TAP) and scan dates;
Baseline CT-TAP RECIST;
Baseline brain CT scans and scan dates;
Baseline PET/CT scans and scan dates;
Baseline brain MRI scans and scan dates;
The extent of metastatic load assessment via baseline imaging;
The extent of metastatic disease at baseline, and the status of affected organs;
First and subsequent evaluations of chest CT scans and scan dates;
First and subsequent evaluations of abdominal CT scans and scan dates;
RECIST-based evaluation criteria for CT scans;
First and subsequent evaluations of CT-TAP scans and scan dates;
First and subsequent evaluations of brain CT scans and scan dates;
First and subsequent evaluations of brain MRI scans and scan dates;
Follow-up imaging after the first/further evaluations, including chest CT scans, CT-TAP scans, and brain CT scans, as well as their corresponding dates; and
Progression evaluation imaging, including chest CT scans, CT scans RECIST criteria, CT-TAP scans, and brain CT scans, and their corresponding dates.

It is understood that radiological data may be collected once at baseline, at subsequent time points, or at multiple intervals during the course of treatment.

Clinical data may include demographic information, such as gender, age, ethnicity, and the like. Clinical data may also include medical history of the cohort of medical subjects 116 or medical subjects 118, such as smoking status, eating habits, physical activity, lifestyle, height, weight, personal history of diseases, previous (familial) history of diseases (including date of start of disease, disease stage, disease status at diagnosis, treatment history, past hospitalizations, and/or death and organs affected, performance status and clinical response at first/further evaluation, progression status, including date and site of progression, treatment status after progression and vital status at most recent update), and the like. In some embodiments, the clinical data may have a temporal component, and may be collected in time series from a given starting point (also referred to as longitudinal data).

Similarly, clinical data for a cohort of medical subjects 116 or medical subjects 118 diagnosed with cancer may include demographics, including, gender, age, ethnicity, etc. Moreover, clinical data for a cohort of medical subjects 116 or medical subjects 118 having cancer may further comprise medical history such as height, weight, smoking status, autoimmune disease history, pre-existing conditions, familial history of cancer, prior personal history of cancer, etc. Yet further, clinical data for a cohort of medical subjects 116 or medical subjects 118 with cancer may also include disease history. Specifically, the disease history may be comprised of data such as cancer subtype, performance status at the time of diagnosis, history of corticosteroid treatment within 12 months of diagnosis, history of antibiotic treatment within one month of diagnosis, therapeutic regimens administered, medication dosing schedules, the number of therapy cycles received (both at first evaluation and by progression), presence of treatment-related toxicities necessitating discontinuation, hospitalization, clinical performance status and response to treatment at initial and subsequent evaluations, progression status (including dates and sites of progression), treatment and therapy following progression, second-line therapies administered, date and status of last available follow-up, and cause of death, if applicable.

As a nonlimiting example, clinical data for a patient with cancer, may include information on age, performance status as per the Eastern Cooperative Oncology Group (ECOG) scale, history of autoimmune diseases, corticosteroid and antibiotic treatment histories, gut microbiome data, and disease history (including liver, brain, and bone metastases), as well as immune-related adverse events.

Moreover, clinical data may include descriptive variables such as the cohort of medical subjects 116 or medical subjects' 118 response to treatment and disease progression. Treatment responses may be categorized as a complete response, partial response, stable disease, or progression, while disease progression may be classified by factors such as increased tumor growth rate or invasiveness. These classifications may be assigned numerical variables as part of a data preprocessing step to facilitate further analysis.

Clinical data may also include survival time, progression-free survival time, and other longitudinal quantitative variables.

Biological data may include data on laboratory/clinical tests and/or examinations (such as blood tests, biopsies, etc.), metabolomics, proteomics, pathology data, physiological data (such as heart rates, electrocardiogram, electroencephalogram, etc.), and the like, but not limited thereto. Clinical and biological data may be obtained from databases such as electronic medical records (EMRs), electronic health records (EHRs), personal health records (PHRs) or an electronic case report form (eCRF). Clinical data and biological data may also be obtained from a Laboratory Information Management System (LIMS).

Additionally, biological data may include for the cohort of medical subjects 116 and/or medical subjects 118, the disease type and stage, expression levels of relevant receptors, and blood analyses conducted at baseline and subsequent evaluations (encompassing both hematology and biochemistry).

Furthermore, biological data specific to members of the cohort of medical subjects 116 and/or a medical subject 118 diagnosed with cancer may encompass, the subject's 116/118 cancer stage and histopathological classification at the time of diagnosis, expression levels of relevant receptors, and blood analyses conducted at baseline and subsequent evaluations (encompassing hematology and biochemistry).

As a nonlimiting example, for the cohort of medical subjects 116 or medical subjects 118 diagnosed with cancer, biological data may include, histopathological classification of the cancer at diagnosis, programmed cell death ligand 1 (“PD-L1”) expression levels, the immunohistochemistry antibody employed to measure PD-L1, and blood analyses conducted at baseline and at subsequent evaluations (including hematology and biochemistry).

In an embodiment, biological data for the cohort of medical subjects 116 or medical subjects 118 having cancer, may include information on the expression of PD-L1 on tumor cells. In a further embodiment, biological data for a medical subject 118, particularly those with lung cancer, may comprise neutrophil-to-lymphocyte ratio, enzyme lactate dehydrogenase (LDH) levels, and/or blood tumor mutational burden (bTMB).

In yet a further embodiment, biological data for the cohort of medical subjects 116 or medical subjects 118 with cancer, may consist of the following:
Histopathological classification of the cancer at diagnosis;
PD-L1 expression levels;
Immunohistochemistry antibodies employed to measure PD-L1;
Dates of blood analyses at baseline and subsequent evaluations thereof;
Blood parameters at baseline, including, but not limited to, white blood cell count, neutrophil count, lymphocyte count, monocyte count, eosinophil count, basophil count, platelet count, red blood cell count, hemoglobin levels, LDH levels, albumin levels, and CRP levels; and
Blood parameters at subsequent evaluations, including, but not limited to, white blood cell count, neutrophil count, lymphocyte count, monocyte count, eosinophil count, basophil count, platelet count, red blood cell count, hemoglobin levels, LDH levels, albumin levels, and CRP levels.

Biological data may comprise metabolomic data, obtained for example using mass spectrometry, or proteomic data, obtained for example using mass spectrometry, liquid chromatography or protein assays.

Each of the aforementioned medical and biological data may be obtained from corresponding data sources 110, as described above. The medical data from each of the data sources 110 associated with each member of the cohort of medical subjects 116 or individual medical subject 118 may be aggregated to form the multimodal medical data. The multimodal medical data may be stored in a database, such as database 112, which may be accessible by the system 102 through a network 104. In one embodiment, the system 102 is designed to acquire medical data in the form of data files, encompassing genomic, clinical, radiological, and/or biological data, from various data sources 110. These data files are accepted by the system 102, ensuring compatibility with different formats and standards used in medical data acquisition. Additionally, the system 102 may incorporate the physical pipelines to retrieve or generate such data. For example, the system 102 may include components, assays, hardware, and the like for retrieval of genomic, biological, radiological, and/or clinical information. For example, the system may include the kits necessary for sample capture and subsequent sequencing, which are essential for determining, for example, genomic modality information for a medical subject 118. Once acquired, this data may be stored in the database 112, allowing the system 102 to seamlessly process and analyze data from multiple modalities.

In some embodiments, the multimodal medical data may be collected for multiple cohorts of medical subjects 116, such as for training the predictive models 113. The medical subjects 118 may be grouped into different cohorts, based on the medical data, such as the history of treatment and the like. In some embodiments, the cohorting is performed automatically based on pre-determined criteria that can be extracted from the input genomic, radiomic, clinical or biological data. For example, the medical subjects 118 may be grouped into different cohorts based on the treatment received. In other embodiments, the cohorting is performed automatically as part of the modelling and without pre-determined criteria. For example, the medical subjects 118 may be grouped into different cohorts based on their likely response to a given treatment according to a given multimodal model.

In some embodiments, multiple predictive models 113 may be trained for generating clinical predictions for all or a subset of multimodal medical data. In some embodiments, at least one of the predictive models 113 may be trained for each cohort of medical subjects 116. In other embodiments, multiple predictive models 113 may be trained for determining clinical predictions for the onset and progression of each disease for each cohort of medical subjects 116. In further embodiments, multiple predictive models 113 may be trained for determining multiple clinical predictions on efficacy for different treatment options for each disease in each of the cohorts 116. In further embodiments, multiple predictive models 113 may be trained on multiple cohorts for modelling the response to different treatment options. In further embodiments, multiple predictive models 113 may be trained for identifying subgroups within a cohort that are predicted to respond better to a given treatment option.

The trained predictive model 113 may be stored in a model bank 114.

The trained models may include imputation models, such as K-nearest neighbor, or mice forest. The trained models may also include machine learning models, for example based on random forest, cox, SVM, gradient boosting, or the like. The models may further include neural network models, such as resnet, densenet, transformers, mamba, or the like. The models may further include ensemble models. Each of the trained models may consist of an architecture, a set of parameters, and a set of hyperparameters, stored in one or several machine-readable files, such as one or several plain text files. The model bank 114 may be implemented as a database for storing the models, or as a server configured to execute the predictive models 113 to generate the clinical predictions, among other insights. The model bank 114 may be accessible by the system 102 either directly, or through the network 104. In one embodiment, the trained predictive model 113 may only be stored in the model bank 114 if it has been classified as relevant and/or accurate. Classifying the trained predictive model 113 as relevant and/or accurate may be an automated and/or a manual process.

Once the predictive models 113 are trained, the system 102 may also be configured to receive multimodal medical data associated with individual medical subjects 118 for inference (i.e., to generate clinical predictions for individual medical subjects 118), such as by medical practitioners for determining personalized treatment options for individual medical subjects 118. The request to generate clinical predictions for individual medical subjects 118 may be transmitted by the user(s) 108 using the corresponding computing device 106. The user 108 may be any stakeholder interested in the clinical predictions made for the multimodal medical data associated with one or more of the medical subjects 118. For example, the user 108 may be a healthcare practitioner attempting to diagnose and provide customized treatment options to the medical subjects 118, healthcare administrators for forecasting and prediction of diseases to better equip medical institutions, medical policymakers to understand causes of diseases and prescribe medical policies therewith, medical subjects 118 trying to understand potential risk for diseases, and the like.

The system 102 may be an interoperable inference system, for example, configurable as a software package adapted to reside within a host system. In some embodiments, the host system may be a cloud-based system, such as Microsoft Azure's cloud computing platform. The inference system may be designed to receive data in native formats specific to the host system, thereby eliminating the need for extensive data conversion. The interoperable inference system may integrate via an API or other interfacing means, allowing seamless data exchange between the interoperable inference system and the host system. In an embodiment, the interoperable inference system processes the input data and generates output results in a format compatible with the host system's interpretative and presentation capabilities. This ensures the results are readily interpretable and presentable by the host system. The interoperable inference system may be scalable and modular, enabling efficient operation and flexible deployment across diverse host environments. For the purposes of this disclosure, a “host system” may be any system partially or wholly integrating the inference system.

In another embodiment, the system 102 may be an inference system, for example, configurable as a software package adapted to be accessed by a calling system via a network. For example, a cloud-based system, such as Microsoft Azure's cloud-based computing platform. The inference system resides on a server and is designed to receive data from the calling system in its native formats, eliminating the need for extensive data conversion. The inference system is accessed via an API or other interfacing means, allowing seamless data exchange between the inference system and the calling system. In an embodiment, upon receiving the input data, the inference system processes it and generates output results in a format compatible with the calling system's interpretative and presentation capabilities. This ensures that the results are readily interpretable and presentable by the calling system. For the purposes of this disclosure, a “calling system” may be any system communicating with the inference system.

In some embodiments, the users 108 may access the clinical predictions using an interface on the computing device 106. The computing device 106 may be any one or combination of including, but not limited to, smartphones, mobile phones, desktop computers, laptops, tablets, virtual computers, servers, and the like. The interface may be any one or combination of Graphical User Interfaces (GUI), Application Programming Interfaces (API), Command Line Interfaces (CLI), hardware interfaces, and the like. In such embodiments, the system 102 may be configured to transmit the clinical predictions generated for given multimodal medical data to the computing device 106 for display on the corresponding interfaces. The computing device 106 may be configured to send requests and receive clinical predictions through the network 104. In some embodiments, the computing devices 106 may be integrated with the system 102. In some embodiments, the system 102 may provide clinical predictions on the multimodal medical data on a unified interface.

The network 104 may be any wired or wireless communication network. Examples of wired communication networks may include electrical wires, cables, optical fiber cables, and the like, but not limited thereto. Examples of wireless communication network include communications network capable of transferring data using means including, but not limited to, radio communication, satellite communication, a Bluetooth, a Zigbee, a Near Field Communication (NFC), a wireless fidelity (Wi-Fi) network, a light fidelity (Li-Fi) network, the Internet, a carrier network including a circuit-switched network, a packet switched network, cellular telecommunication networks, combinations thereof, and the like.

While FIGS. 1A and 1B illustrate some of the units/components of the architectures 100A, 100B, it may be appreciated by those skilled in the art that other components/units may be suitably adapted and included in the architectures 100A, 100B for the operation of the system 102. Further, the present disclosure describes a few arrangements of components/units of the architectures 100A, 100B in FIGS. 1A and 1B, however, it may be appreciated by those skilled in the art that embodiments of the present disclosure may be suitably adapted to have different arrangements of the components/units for the operation of the system 102.

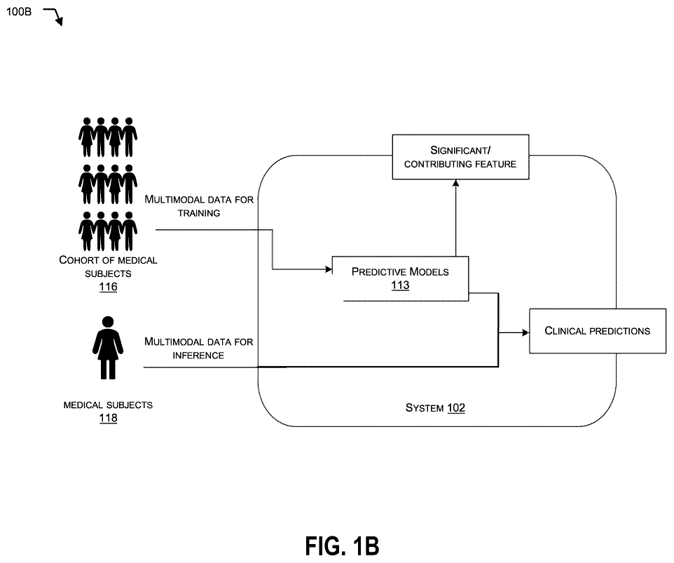

Various components and operations of the system 102 are described in detail in reference to FIG. 2. Referring to block diagram 200 in FIG. 2, the system 102 may include one or more processor(s) 202. The one or more processor(s) 202 may be implemented as one or more microprocessors, microcomputers, microcontrollers, digital signal processors, central processing units, logic circuitries, and/or any devices that manipulate data based on operational instructions. Among other capabilities, the one or more processor(s) 202 may be configured to fetch and execute computer-readable instructions stored in a memory 204. The memory 204 may store the computer-readable instructions or routines, which may be fetched and executed to create or share the data units to other elements of the system 102. The memory 204 may include any non-transitory storage device including, for example, volatile memory such as Random Access Memory (RAM), or non-volatile memory such as an Erasable Programmable Read-Only Memory (EPROM), flash memory, and the like.

In an embodiment, the system 102 may also include an interface(s) 206. The interface(s) 206 may include a variety of interfaces, for example, interfaces for data input and output (I/O) devices, referred to as I/O devices, storage devices, and the like. The interface(s) 206 may facilitate communication between the system 102 and the database 112, the model bank 114, and the computing device 106 using peripherals allowing wireless communication using the network 104. The interface(s) 206 may also provide a communication pathway for one or more components within the system 102. Examples of such components include, but are not limited to, processing engine(s) 208 and database 112.

While FIG. 1A shows embodiments where the database 112 and the model bank 114 are external to the system 102, in other embodiments, such as those shown in FIG. 2, the database 112 and the model bank 114 may be implemented within the system 102. The database 112 may also include data that is either stored or generated as a result of functionalities implemented by any of the components of the processing engine(s) 208, aside from multimodal medical data received from multiple data sources 110. Further, the model bank 114 may include the plurality of predictive models 113 trained by the system 102.

In an embodiment, the processing engine(s) 208 may be implemented as a combination of hardware and software (for example, programmable instructions) to implement one or more functionalities of the processing engine(s) 208. In examples described herein, such combinations of hardware and software may be implemented in several different ways. For example, the software for the processing engine(s) 208 may be processor-executable instructions stored on a non-transitory machine-readable storage medium and the hardware for the processing engine(s) 208 may include a processing resource (for example, one or more processors), to execute such instructions. In other embodiments, the processing engine(s) 208 may be implemented by electronic circuitry.

In some embodiments, the processing engine(s) 208 may include a data management engine 210, a training engine 212, an inference engine 214, and other engine(s) 216. The other engine(s) 216 may implement functionalities that supplement applications/functions performed by the system 102.

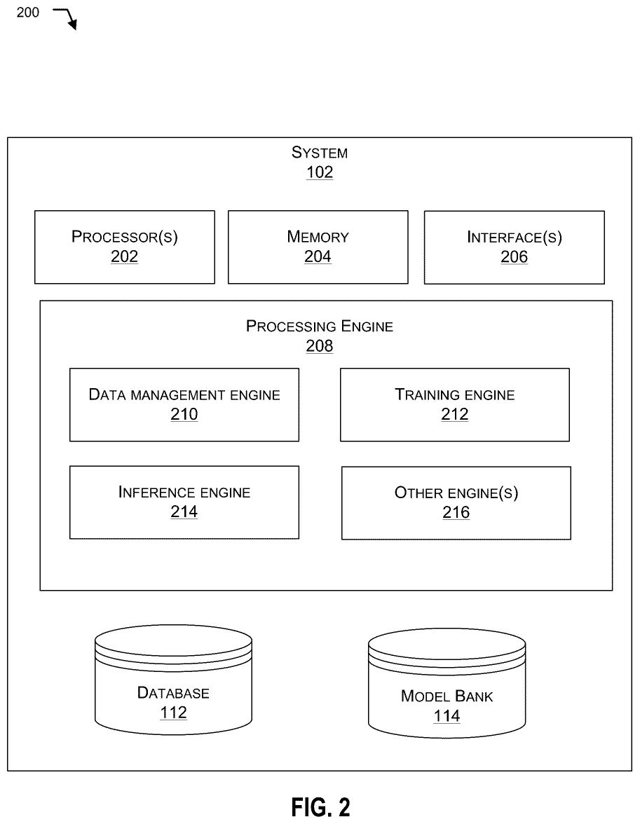

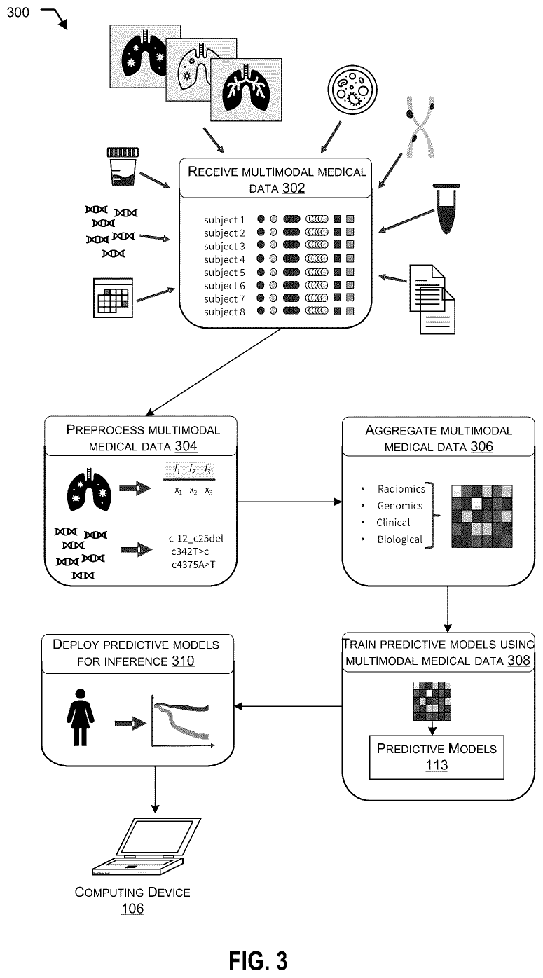

In some embodiments, the data management engine 210 and the training engine 212 may be configured to implement the method 300 shown in FIG. 3. In some embodiments, the inference engine 214 may be configured to implement the method 400 shown in FIG. 4.

### Training

Referring to FIG. 3, a method 300 for training predictive models to generate clinical predictions from multimodal medical data may be executed by the data management engine 210 and the training engine 212.

As shown, at step 302, the method 300 may include receiving/ingesting/retrieving multimodal medical data from the data sources 110. In some embodiments, the data management engine 210 may be configured to receive the multimodal medical data as a dataset, which may be stored in the database 112. In other embodiments, the data management engine 210 may be configured to receive the multimodal medical data in real-time or near-real time, such as when the multimodal medical data may be received from medical subjects 116 involved in a clinical trial, as the clinical trial is being conducted. In some embodiments, the data management engine 210 may be configured to mine for an ensemble of medical subjects 116 with subject-level data, for instance belonging to previous cohorts or multimodal medical data provided to the system 102 to obtain clinical predictions (i.e., during inference), for training the predictive models 113. The multimodal medical data may be received in different formats from different data sources 110. The data management engine 210 may be configured to use different sub-engine/modules to parse the data received in different formats, and encode the data for training the predictive models 113, as described subsequently in the present disclosure.

In one embodiment, the data management engine 210 may further comprise a community database comprising the subject-level data. The community database may be utilized to refine any of the predictive models 113 and/or produce new predictive models 113.

In some embodiments, the multimodal medical data may be received as multiple sets of unimodal medical data, such as gene sequences, clinical data, and PET scans of each medical subject 116 participating in a clinical trial, for example. In such embodiments, the data management engine 210 may be configured to reconcile the multiple unimodal medical data received into the multimodal medical data. For example, the data management engine 210 may be configured to identify the medical subject 116 to whom/which the unimodal medical data is associated with, and assign the corresponding unimodal medical data to the identified medical subject. The data management engine 210 may store the reconciled (now multimodal) medical data in the database 112 corresponding to each of the medical subjects 116. For clarity, in many embodiments, data derived from medical subjects 116 may be utilized in training and developing one or more of the models, while data derived from subjects 118 may be routed through one or more of these aforementioned models. In some embodiments, the data management engine 210 may store in the database 112 the data, which may be raw data or reconciled data, corresponding to additional subjects 118 who have been inputted into the multimodal factory after the training of the initial models. In some embodiments, the database 112 may have multiple levels, as some types of medical data (such as MRI images) may be represented using multiple files. In some embodiments, the multimodal medical data stored in the database 112 may be a labelled dataset having treatment outcomes of the cohort of medical subjects 116 as labels.

In some embodiments, the data management engine 210 includes tools to perform automatic quality checks as part of step 304. As a non-limiting example, images that are of insufficient quality, data values that fall outside of plausible ranges determined based on observed distributions in databases, data that are in an incorrect format, inconsistent metadata information such as dates, and the like, are identified and removed.

In some embodiments, the data management engine 210 is configured to automatically classify images, determining which tissues are visible in the picture and/or which method was used to generate the image, including the injection time. As a non-limiting example, image classification may use machine learning models previously trained on databases of medical images. In some embodiments, the classification is reported as metadata of the image or as part of a database incorporating the images.

In some embodiments, the data management engine 210 is configured to handle and reroute images. As a non-limiting example, the data management engine 210 may be used to query stored images and retrieve images corresponding to a given patient, tissue, or imaging type based on their metadata or information from a database, either provided with the original images or automatically inferred from the images. As a further non-limiting example, the data management engine 210 may be used to assign images to different time points of the medical journey of the subject, such as in relation to a treatment. The retrieved images may then be integrated in the at least one dataset generated for the cohort of medical subjects 116 as part of step 304. The data management engine 210 may be configured to assign the same image to multiple tissues, if those are visible therein.

In some embodiments, the data management engine 210 may also be configured to convert/encode the representations of the multimodal medical data into either numerical or categorical variables. For example, gene sequences may be converted into 4 Boolean features, where each Boolean feature corresponds to a nucleotide. In another example, pixels in the images in the radiological data may be converted into numeric features. Further, clinical data, such as gender, ancestry, presentation of symptoms, and the like, may be represented as categorical features (using Boolean, categorical, or ordinal variables/labels). In some embodiments, continuous variables are centered-normalized. In some embodiments, outlier values are replaced, for example by defaulting values below the 1% or 99% percentiles to the value of the 1% or 99% percentile.

At step 304, the method 300 may include preprocessing the multimodal medical data. To implement the step 304, the data management engine 210 may be configured to perform any one or a combination of reconciling multiple unimodal medical data, imputing missing values in the multimodal medical data, cleaning errors in the multimodal medical data, extracting/identifying one or more features from the plurality of medical data and selecting a subset of features associated with a condition of interest, and the like, but not limited thereto. In an embodiment, the condition of interest may be evaluated by an expert in the field, such that said expert may determine which data points are informative for a given condition of interest. In a further embodiment, the set of features informative for the condition of interest may be automatically determined, for example based on previously analyzed datasets. In such an embodiment, the automatic determination of the set of features informative for the condition of interest may be informed by a referential database or a database containing previous analyses. In some embodiments, the data management engine 210 may be configured to perform data imputation and cleaning using techniques known to those skilled in the art, such as linear interpolation, mean imputation, median imputation, mode imputation, constant imputations, K-nearest neighbor imputations, deletions, removing duplicates, standardization, normalization, and the like.

In some embodiments, the data management engine 210 may be configured to extract/identify features of interest from the multimodal medical data, for example, as part of step 304. For example, the data management engine 210 may be configured to process images, automatically segmenting the images and extracting features, such as shape, intensity, and texture, or the like. In another example, gene sequence data, in the form of high-throughput sequencing reads, methylation profiles, DNA microarray data, RNA microarray data, Sanger sequence, or the like, using a form of a bioinformatic pipeline or biostatistic tool to identify genetic variants, assess genomic profiles, establish gene expression patterns, extract other genomic features, estimate tumor content, or the like. Other types of automatic data preprocessing performed by the data management engine 210 may include extraction of metabolomic or proteomic data from biological data. In other embodiments, once the multimodal medical data is encoded, the data management engine 210 may be configured to select significant features using techniques known to those skilled in the art, such as Recursive Feature Elimination (RFE), Exhaustive Feature Selection, correlation coefficients, Principal Component Analysis (PCA), Linear Discriminant Analysis (LDA), Simulated Annealing, Cross-validation based selection, and the like, but not limited thereto. In some embodiments, different sets of features may be selected for different diseases studied on the cohort of medical subjects 116, and treatment outcomes therefor.

At step 306, the method 300 includes aggregating one or more features of the multimodal medical data. In some embodiments, the data management engine 210 may be configured to aggregate the multimodal medical data of the medical subjects in the cohorts 116 successively (i.e., aggregating features of a first modality and then aggregating features of other modalities). In some embodiments, the aggregation of different modalities can include assigning, by manual or automatic methods, distinct weights to each modality, to compensate for some modalities having a potential explanatory power inflated or deflated due to the data structure. As a non-limiting example, clinical data such as cancer type, treatment type, and the like, encompassing a wealth of information in few variables may be weighted down. As another non-limiting example, genomic data, such as genetic variants detected across the genome or expression level of each of thousands of genes, with potential explanatory power spread across numerous variables, may be weighted up. In some embodiments, the aggregated features may include the features extracted during feature extraction. In other embodiments, the aggregated features may include features selected/curated manually, based on the features that are available in clinical settings.

At step 308, the method 300 includes training one or more of the predictive models 113 based on the aggregated features of the multimodal medical data to generate clinical predictions. The step 308 may be executed by the training engine 212. In some embodiments, the predictive models 113 may be any or a combination of machine learning models, symbolic reasoning models, or statistical models. Examples of machine learning models include linear regression, logistic regression, Bayesian classification models, support vector machines, decision trees, random forests, neural networks, convolutional neural networks, recurrent neural networks, encoder-only models, decoder-only models, encoder-decoder models, transformer models, and the like. The size of the models may be determined based on the size of the multimodal medical data of the cohorts of medical subjects 116. Examples of symbolic reasoning models include expert systems, rule-based systems, knowledge representations, logic programming, ontology-based systems, and the like. Examples of statistical systems include time series models (such as Auto-Regressive Integrated Moving Average (ARIMA)), Bayesian models, Markov models, Analysis of Variance (ANOVA), Principal Component Analysis (PCA), cluster analysis, and the like. The models may further include neural network models, such as resnet, densenet, transformers, mamba, or the like. In some embodiments, an ensemble of models may be trained for generating the clinical predictions using the multimodal medical data. The predictive models 113 may be trained using techniques known the art, such as backpropagation, Generative Adversarial Network (GANs) training, multi-task training, reinforcement training, and the like, to generate the clinical predictions.

In some embodiments, model training involves selecting the features to include in the model. Possible methods to select features include clustering, univariate feature selection, correlation-based feature selection, variance thresholding, recursive feature elimination, or variable importance feature selection. In some embodiments, parameters for the inclusion of features are optimized during the training of models.

In some embodiments, the predictive models 113 may also be incorporated into autonomous agents. The autonomous agents may be configured to execute other processor-executable instructions based on inputs provided thereto. In some embodiments, the autonomous agents may be configured to coordinate the use of multiple machine learning models, symbolic reasoning models, and/or statistical models to generate the clinical predictions. In some embodiments, the autonomous agents may be configured to retrieve data from external sources (such as when the multimodal medical data is incomplete) by making API calls, invoking functions associated with different software libraries, executing command line functions, and the like, and generate the clinical predictions therewith.

In some embodiments, at least one predictive model 113 may be trained to determine at least one of risk of the disease, progression of the diseases, progression-free survival time, overall survival time, efficacy of different treatments on the disease, treatment outcome (such as survival time), treatment benefit, occurrence of adverse events, best clinical response, and the like. In some embodiments, at least one predictive model 113 may be trained for each disease for each cohort of medical subjects 116 in the clinical predictions. In some embodiments, placebo and “no treatment” may also be included in the treatment options, as a control group/model. In some examples, the clinical predictions generated may indicate progression of the disease in time series when a particular treatment option is provided. In other examples, the clinical predictions may indicate occurrence of adverse events for the treatment option provided to a particular cohort of medical subjects 116. It will be apparent to those skilled in the art that any outcome for which data exists for individuals from the cohort of medical subjects 116 can be predicted using the described invention. In some implementations, the system may process and analyze data from multiple diseases or conditions simultaneously. For example, the system may pool data from different types of cancers, such as kidney cancer and lung cancer, into a single dataset for model development. This approach may allow for the creation of a unified predictive model that can generate clinical predictions across various cancer types, potentially revealing shared prognostic factors or treatment response patterns that might not be apparent when analyzing each cancer type in isolation. In such instances, the model may be configured to ‘ignore’ cancer types (or other conditions) not relevant to the instant task.

In some embodiments, performance of the predictive models 113 may be assessed. In some embodiments, a training set and a test set may be partitioned from the multimodal medical data, where the training set may be used for training the predictive models 113, and the test set may be used for testing performance of the predictive models 113. In some embodiments, a nested cross-validation may be used to optimize and/or test the performance of the predictive models 113. In other embodiments, other optimization techniques, loss functions, hyperparameters, and the like, may be selected for improving the accuracy of the predictive models 113.

In some embodiments, the contribution of the individual features to the clinical predictions may be assessed. The individual features may belong to modalities of medical data associated with the cohort of medical subjects 116. To assess the contribution of features, the method 300 includes randomly shuffling values associated with one of the features in the cohort of medical subjects 116 to generate a pseudo-replicate dataset for each feature included in the model. As a non-limiting example, shuffling of values can be performed using the Fisher-Yates shuffling algorithm, or the like. In such embodiments, values of other features of the medical subjects in the cohort 116 are retained/unchanged. The process is repeated multiple times to obtain, for each feature, a number of pseudo-replicate datasets. As a non-limiting example, a total of 50, 100, 500, or 1000 pseudoreplicates may be generated. The method 300 then includes testing performance of the predictive model 113 on each of the pseudo-replicate datasets. The contribution of the feature to the predictive models 113 is assessed as the decrease of performance resulting from the random shuffling of values, summarized among pseudo-replicates, with estimators of variance. In some embodiments, a report may be produced and transmitted to the computing device 106 describing the predictive models 113, and the contribution of individual features. In some embodiments, the report may be used by entities operating clinical trials, regulatory agencies, medical policymakers, and the like. In an embodiment, the report may be displayed to the user on a computer interface. For example, the report may be downloaded and/or displayed on the computing device 106.

In some implementations, the system may employ various shuffling algorithms to assess feature importance in predictive modeling. These algorithms may involve randomly reassigning the values of a specific feature among individuals in the dataset, creating pseudoreplicates that maintain the overall distribution of the feature while disrupting its relationship with the outcome. The number of pseudoreplicates generated may vary, ranging from a single instance to thousands or more, with, as a nonlimiting example, the accuracy of the contribution estimate and the estimator of variance generally increasing as the number of pseudoreplicates grows. For example, the system may generate 100, 1000, or even 10,000 pseudoreplicates for each feature, allowing for a robust assessment of how changes in the feature's distribution impact the model's predictive performance across the entire dataset.

At step 310, the method 300 includes deploying the predictive models 113 to a model bank 114. The predictive models 113 may be transmitted to the model bank 114, which may store and execute the predictive models 113 on demand. For example, for any requests (having multimodal medical data of the individual medical subject 118 received from the computing device 106, the system 102, using the predictive models 113 in the model bank 114, may generate clinical predictions for the request.

### Inference

The system 102 may be configured to use the inference engine 214 for generating the clinical predictions for multimodal medical data of the individual medical subjects 118. The inference engine 214 may be configured to execute a method 400 for generating the clinical predictions, based on the multimodal medical data.

As shown in FIG. 4, at step 402, the method 400 includes receiving multimodal medical data associated with an individual medical subject, such as the medical subject 118. The multimodal medical data of the medical subject 118 may be transmitted by the user 108 using corresponding computing device 106. As stated, the user 108 may be any stakeholder interested in the clinical predictions for the multimodal medical data of the medical subject 118, such as a medical practitioner intending to provide personalized treatment options. In some embodiments, the inference engine 214 may be configured to receive the multimodal medical data. The system 102 may be configured to provide a unified interface for the users 108 to obtain clinical predictions for any combination of multimodal medical data associated with the medical subjects 118.

In some embodiments, the data management engine 210 includes tools to perform automatic quality checks of the data received, for example, as part of step 402. As a non-limiting example, images that are of insufficient quality, data values that fall outside of plausible ranges determined based on observed distributions in databases, data that are in an incorrect format, and the like, are identified and may be flagged with a warning or removed.

In some embodiments, the data management engine 210 is configured to automatically classify images, determining which tissues are visible in the picture and/or which method was used to generate the image. As a non-limiting example, image classification may use machine learning models previously trained on databases of medical images. In some embodiments, the classification is reported as metadata of the image or as part of a database incorporating the images. In some embodiments, image classification is used to verify information provided by the users 108 as part of the quality checks performed by the data management engine 210 during step 402.

In some embodiments, the data management engine 210 may be configured to convert/encode the representations of the multimodal medical data from the medical subjects 118 into either numerical or categorical variables at step 402. For example, gene sequences may be converted into 4 Boolean features, where each Boolean feature corresponds to a nucleotide. In another example, pixels in the images in the radiological data may be converted into numeric features. Further, clinical data, such as gender, ancestry, presentation of symptoms, and the like, may be represented as categorical features (using Boolean, categorical, or ordinal variables/labels). In some embodiments, continuous variables are centered-normalized. In some embodiments, outlier values are replaced, for example by defaulting values below the 1% or 99% percentiles to the value of the 1% or 99% percentile.

In some embodiments, the data management engine 210 may be configured to perform data imputation and cleaning of the multimodal data from the medical subjects 118 as part of step 402 using techniques known to those skilled in the art, such as linear interpolation, mean imputation, median imputation, mode imputation, constant imputations, K-nearest neighbor imputations, deletions, removing duplicates, standardization, normalization, and the like.

In some embodiments, the data management engine 210 may be configured to extract/identify features of interest from the multimodal medical data of the medical subjects 118 at step 402. For example, the data management engine 210 may be configured to process images, automatically segmenting the images and extracting features, such as shape, intensity, and texture, or the like. In another example, gene sequence data, in the form of high-throughput sequencing reads, methylation profiles, DNA microarray data, RNA microarray data, Sanger sequence, or the like, using a form of a bioinformatic pipeline or biostatistic tool to identify genetic variants, assess genomic profiles, establish gene expression patterns, extract other genomic features, estimate tumor content, or the like. Other types of automatic data preprocessing performed by the data management engine 210 may include extraction of metabolomic or proteomic data from biological data.

In some embodiments, the step 404 may be iterated for each available treatment option for each of the diseases. At step 404, the method 400 may include, for at least one treatment option or absence of treatment, generating at least one clinical prediction based on the multimodal medical data using a corresponding predictive model. At step 404, the method 400 may include generating at least one clinical prediction based on the multimodal medical data using a corresponding predictive model 113. In some embodiments, the inference engine 214 may be configured to receive and preprocess the multimodal data, such as either by using the data management engine 210 or by using techniques used by the data management engine 210. The inference engine 214 may be configured to analyze the multimodal medical data, and select/retrieve the appropriate predictive models 113 from the model bank 114. The appropriate predictive models 113 may be those predictive models 113 that correspond to the disease and the treatment option being explored for the medical subject 118. In some embodiments, the appropriate predictive models 113 are selected and/or selectable by the user 108. In some embodiments, the appropriate predictive models 113 are selected by the data management engine 210 based on the disease indicated by the users 108 and the data received in step 402. As a non-limiting example, different models for a given disease might be appropriate depending on the data available to the users 108. In some embodiments, the most appropriate models 113 are automatically selected by the data management engine depending on the multimodal data inputted by the users 108. As a non-limiting example, the appropriate models might be selected in a disease-agnostic manner depending, for example, on the imaged tissue inputted by the users 108.

In some embodiments, the system may offer multiple approaches for model selection. While one configuration may allow users to manually select the appropriate model, alternative methods may be employed. For instance, the system may implement an automated model selection process based on the available input data. In this scenario, if both genomic and imaging data are present for a given disease, the system might automatically choose a comprehensive model (e.g., Model A) that incorporates both data types. Conversely, if only imaging data is available, the system may default to a different model (e.g., Model B) optimized for image-based predictions. Additionally, the system may incorporate disease-agnostic models that can generate predictions based on specific data types, regardless of the underlying condition. For example, upon detecting lung images in the input data, the data management engine may automatically select a model designed to assess cancer risk from lung imaging, without requiring explicit disease specification. These adaptive model selection approaches may enhance the system's flexibility and broaden its applicability across various clinical scenarios and data availability contexts.

The inference engine 214 may be configured to execute the appropriate predictive models 113, or cause the appropriate predictive models 113 to be used/executed (such as at the model bank 114), to generate the clinical predictions.

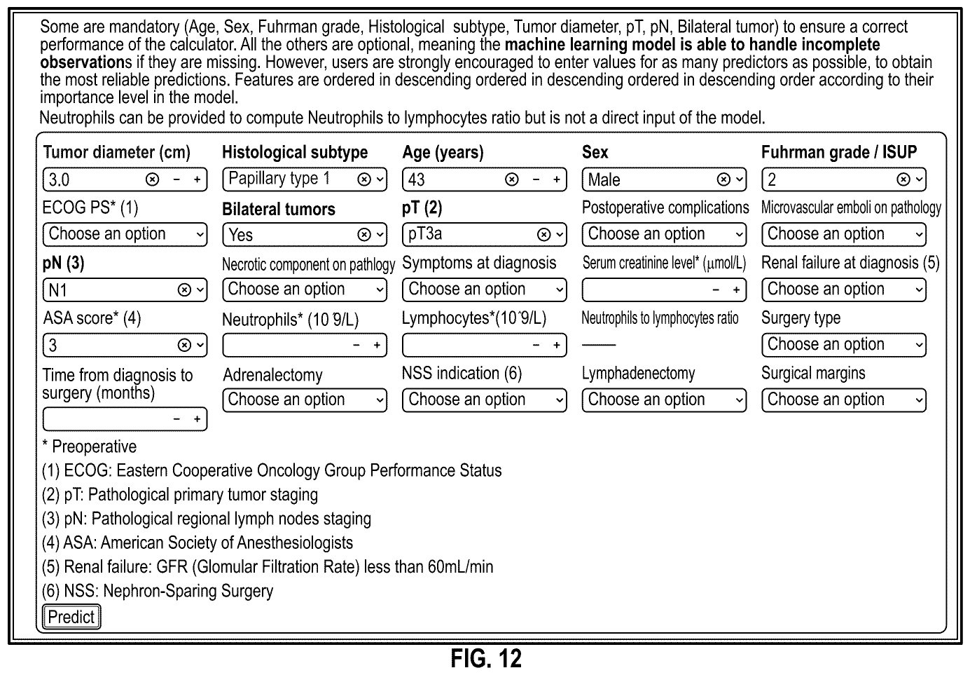

The following is an exemplary workflow depicting how the developed model(s) may be utilized to predict the risk estimator of new subjects, for which some of the features, potentially belonging to different modalities, are available. First, the model may be selected from the model bank 114 based on the investigated condition and/or the goals of the user. In such an instance, data corresponding to features included in the selected model are collected, potentially using a user interface. In such an example, data may be input via a user-facing interface or via an interface facing another computerized element (e.g., a database or third-party system). A non-limiting example of such a user-facing interface, deployed in the case of a study on kidney cancer (Boulenger de Hauteclocque et al. 2023, BJU International 132:160-169, “Machine-learning approach for prediction of pT3a upstaging and outcomes of localized renal cell carcinoma (UroCCR-15)”; Margue et al. 2024, NPJ Precision Oncology 8, 45 “UroPredict: Machine learning model on real-world data for prediction of kidney cancer recurrence (UroCCR-120)”), is provided in FIGS. 12-14. In some embodiments raw data may be automatically processed to extract features. The set of features, including but not limited to those potentially extracted automatically, is assembled. In some embodiments, missing data is imputed. The subject features may be inputted in the selected predictive model to generate predictions of the risk estimator of interest for the subject. In some embodiments, the predictions are repeated with varying conditions, such as different possible treatments. As nonlimiting examples, the risk estimator may be the likelihood of a given action happening (e.g., percent likelihood of a cancer recurrence) or a survival likelihood.

At step 406, the method 400 may include transmitting the at least one prediction for the at least one treatment option or absence of treatment to a computing device of the medical subject. At step 406, the method 400 may include transmitting the at least one clinical prediction for each available treatment option to the computing device 106 of the user 108/medical subject 118. In some examples, the clinical predictions for different treatment options may indicate the potential treatment outcome for the medical subject 118, which may be used to accordingly select and plan the treatment option for the medical subject 118. In other examples, the clinical predictions may indicate the benefit of a given treatment option for the medical subject 118. In yet other examples, the predictions are made for multiple time points, providing time curves of survival or progression-free survival, for each subject and each treatment option.

In some embodiments, the method 400 may include determining a contribution of each feature in the multimodal medical data of the medical subject 118. In such embodiments, the contribution of the features in the multimodal medical data may be determined by, for each feature, randomly varying a value associated with the feature in the multimodal medical data to obtain a corresponding pseudo-replicate data. The value may be varied using a predetermined function. The method 400 may further include generating at least one pseudo-replicate prediction based on the pseudo-replicate data using the corresponding predictive model 113. The contribution (which may be a value or a rank) of each feature may be determined based on difference between the pseudo-replicate prediction and the clinical prediction. A larger difference may indicate that the feature has a higher contribution to the clinical prediction, and vice-versa. The number of pseudoreplicates may be increased to any value such as 50, 100, 500, or 1000, to provide estimates of variance.

### Practical Applications

The system 102 of the present disclosure may be implemented as a platform to generate clinical predictions from multimodal medical data. The use of multimodal medical data improves accuracy of the clinical predictions, in contrast with existing unimodal models. Further, deploying the predictive models 113 on the model bank 114 improves accessibility and useability of the predictive models 113. The system 102 may provide a unified interface to allow users 108 to obtain clinical predictions for any combination of multimodal medical data for the individual medical subject 118. For example, the system 102 may be used by different stakeholders in the health sector, ranging from health practitioners focusing on individual subjects to entities conducting and/or analyzing large clinical trials.

In some implementations, results from clinical trials or real-world evidence may be analyzed to test for efficacy of a given treatment for a disease on a particular cohort of medical subjects 116. Multimodal medical data for two or more cohorts 116 having received different treatments may be provided to the system 102. After data ingestion, reconciliation, processing, and aggregation of the multimodal medical data, the system 102 may train multiple predictive models 113. The contribution of the different features to the clinical predictions may be identified by the system 102 using the predictive models 113 (such as by determining which features contribute positively or negatively to treatment outcomes predicted in the clinical predictions), thereby allowing the user 108 to identify the characteristics that are most important for a likely benefit of the treatment. It is contemplated that the system 102 may become more accurate over time, as it receives more multimodal medical data to train the predictive models 113.

In other implementations, the likely outcome (such as progression-free survival months) of medical subjects 118 suffering from a disease like cancer may be predicted based on a combination of the multimodal medical data associated with the medical subject 118, which, for example, may include a combination of CT scans, blood analyses, and sequence data of the tumor causing the cancer. The features of the multimodal medical data may be provided to the system 102, which may use corresponding predictive models 113 to generate clinical predictions. The clinical predictions may be generated for treatment options where a given treatment is administered to the medical subject 118, and also for the treatment option where the treatment is not administered, thereby allowing the user 108 to determine the predicted efficacy of the treatment to the medical subject 118 based on the clinical predictions.

In other implementations, the models 113 can be trained in step 308 to directly predict the benefit of the treatment and to identify subsets of the cohort 116 most likely to benefit from a treatment. Health practitioners can therefore identify patients that might benefit from a new treatment, with benefits outweighing potential adverse consequences. In such an implementation, the trained models 113 can then be deployed in step 310 and a user interface (for example, those shown in FIGS. 12-14) can allow any health practitioners to obtain predictions of the treatment effect for any new patients 118.

In effect, the system may enhance the quality of care delivered to patients by accurately assessing and optimizing treatment protocols based on real-time data and advanced analytics. This system enables healthcare providers to tailor treatments to individual patient needs, thereby increasing the likelihood of recovery and positive health outcomes. By leveraging precise and personalized treatment recommendations, the system significantly improves patient management and clinical decision-making processes.

While the embodiments of the present disclosure are described in the context of generating clinical predictions from multimodal medical data, it may be appreciated by those skilled in the art that the system 102 may be suitably adapted for other applications. In some embodiments, the system 102 may be configured to generate the clinical predictions using unimodal medical data, such as when multimodal medical data is not available. In other embodiments, the system 102 may be configured to allow for backdoor addition of modalities for applications that are otherwise unimodal.

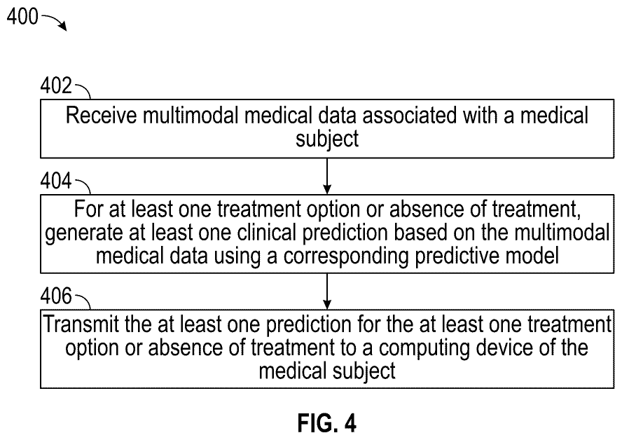

The system 102 and the methods 300 and 400 may be implemented on a computer system. Referring to FIG. 5, the block diagram represents a computer system 500 that includes an external storage device 510, a bus 520, a main memory 530, a read only memory 540, a mass storage device 550, a communication port 560, and a processor 570. A person skilled in the art will appreciate that the computer system 500 may include more than one processor 570 and communication port 560. The processor 570 may include various modules associated with embodiments of the present disclosure. The communication port 560 may be any of a Recommended Standard 232 port for use with a modem-based dialup connection, a 10/100 Ethernet port, a Gigabit or 10 Gigabit port using copper or fiber, a serial port, a parallel port, or other existing or future ports. The communication port 560 may be chosen depending on a network, such as a Local Area Network (LAN), a Wide Area Network (WAN), or any network to which computer system 500 connects.

In an embodiment, the memory 530 may be a RAM, or any other dynamic storage device commonly known in the art. The Read-Only Memory (ROM) 540 may be any static storage device(s) e.g., but not limited to, a Programmable Read-Only Memory (PROM) chip for storing static information. The mass storage 560 may be any current or future mass storage solution, which may be used to store information and/or instructions. Exemplary mass storage solutions may include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), one or more optical discs, Redundant Array of Independent Disks (RAID) storage, e.g., an array of disks (e.g., SATA arrays).

In an embodiment, the bus 520 communicatively couples the processor(s) 570 with the other memory, storage, and communication blocks. The bus 520 may be, e.g., a Peripheral Component Interconnect (PCI)/PCI Extended (PCI-X) bus, Small Computer System Interface (SCSI), USB, or the like, for connecting expansion cards, drives, and other subsystems as well as other buses, such a front side bus (FSB), which connects the processor 570 to the computer system 500.

In another embodiment, operator and administrative interfaces, e.g., a display, keyboard, and a cursor control device, may also be coupled to the bus 520 to support direct operator interaction with computer system 500. Other operator and administrative interfaces may be provided through network connections connected through communication port 560. In some embodiments, the external storage device 510 may be any kind of external hard-drives, floppy drives, Compact Disc-Read Only Memory (CD-ROM), Compact Disc-Re-Writable (CD-RW), Digital Video Disk-Read Only Memory (DVD-ROM). Components described above are meant only to exemplify various possibilities. In no way should the aforementioned exemplary computer system 500 limit the scope of the present disclosure.

While the foregoing describes various embodiments of the present disclosure, other and further embodiments of the present disclosure may be devised without departing from the basic scope thereof. The scope of the present disclosure is determined by the claims that follow. The present disclosure is not limited to the described embodiments, versions or examples, which are included to enable a person having ordinary skill in the art to make and use the present disclosure when combined with information and knowledge available to the person having ordinary skill in the art.

### Treatment Outcome Prediction

The present disclosure contemplates the development of a particular computer-implemented method to predict treatment response or treatment efficacy for a specific patient based on individual-level multivariate data. More particularly, the present disclosure contemplates a statistical approach to improve prediction of the response of individuals to a treatment based on individual-level multivariate data (e.g., genomic, biological, clinical, radiological). The new statistical approach can be applied to the outcome of studies (e.g., clinical trial or real-world data), where individuals can be divided in at least two groups, the first of which received a given treatment while the second did not or received a distinct treatment, and where the health outcome of the individuals was measured, for example as the survival time.

The present disclosure contemplates the use of machine learning to predict the health outcome based on the multimodal data, independently for the group that received the treatment of interest and the group that did not receive the treatment. These two predictions are then combined to obtain an effect of the treatment in terms of the measured health indicator for each subject. Thus, the model optimization is performed on the effect of treatment and not on the outcome predictions for each treatment. Model optimization based on a single variable instead of two variables strongly reduces the sources of errors, improving the overall predictions and the capacity to identify subjects most likely to benefit from a given treatment.

By optimizing a single model based on the difference between the predicted outcomes with versus without treatment as opposed to optimizing independently models for the outcome with treatment and for the outcome without treatment, the invention decreases the sources of errors when predicting the individual-level response to a given treatment. It therefore provides an improved statistical framework to obtain individualized predicted benefits based on data for individuals having either received the treatment or not received the treatment/received a distinct treatment.

It is an object of the systems and methods described herein to provide a statistical approach to predict, with more accuracy at the individual level, the benefit of receiving a given treatment compared to a reference based on data for subjects that have either received a given treatment or not received the treatment/received a distinct treatment.

A method for identifying subjects most likely to respond to a given treatment among subjects having a condition is provided. The condition may be a medical condition, such as cancer or a disease. Of course, other conditions are contemplated and the aforementioned are provided as nonlimiting examples only. The method may provide a statistical approach to improve prediction of the response of subjects to a treatment based on a series of feature data.

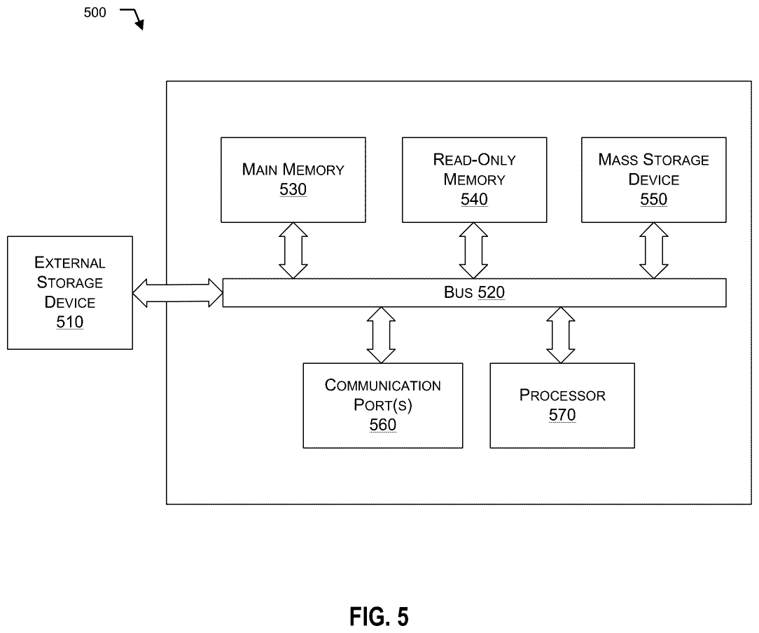

FIG. 6 illustrates a graph representing a progression of a health indicator, such as a survival probability, for two groups of diseased subjects, one of which received a given treatment. Instead of modelling the outcome for each group (demonstrated as the black and dashed curves shown in FIG. 6), as is traditionally done, before inferring the treatment response as the difference between the two (demonstrated as the double-headed vertical arrow in FIG. 6), the difference between the two groups is directly modelled as a response variable.

FIG. 6 illustrates the innovative approach of the methods and systems described herein, which employs machine learning to predict health outcomes based on multimodal data for both the treatment arm and the control arm independently. The graph visually represents the progression of a health indicator over time for two groups: one receiving the treatment and the other not. The solid curve depicts the health indicator for the treatment group, while the dashed curve represents the control group. The vertical double-headed arrow indicates the treatment effect, calculated as the difference between the two curves. This method focuses on optimizing the model based on the treatment effect, rather than separately optimizing predictions for each group. By concentrating on a single variable—the treatment effect—this approach significantly reduces error sources, enhancing the accuracy of predictions and improving the ability to identify individuals who are most likely to benefit from the treatment. This streamlined optimization process offers a more precise and reliable framework for personalized treatment efficacy assessment.

The method may comprise receiving data for a first cohort of subjects and a second cohort subjects, each cohort of subjects comprising a plurality of data. In one embodiment, the first cohort may be a treatment arm wherein the plurality of data corresponds to a plurality of subjects receiving a treatment and the second cohort may be a control arm wherein the plurality of data corresponds to a plurality of subjects not receiving the treatment. Each of the first and second arms may comprise a treatment condition, a variable to assess the health of the subjects, and the series of feature data. The variable to assess the health of the subjects is collected at least two times, for example, at least once before the treatment and at least once after the treatment. The series of feature data may be any features contemplated by a person of ordinary skill in the art, including, without limitation, biological, clinical, imaging, radiological, genomic, or other data. In one embodiment, the series of feature data comprises pre-processed data. The pre-processed data may replace outliers, center-normalize numerical variables, and transform categorical data variables with numerical values. Further, in some embodiments, data imputation may be used to assign values for features in the series of feature data not characterized in some of the subjects.

The method may be a computer-implemented method comprising a machine learning model to train a dataset. The machine learning model may be any suitable machine learning model known to those of ordinary skill in the art. Any of the plurality of data may be used to train the dataset. In some embodiments, any of the pre-processed data may be utilized to train the dataset. In a further embodiment, the dataset may be trained by any of the data, wherein the data or portions thereof may be subject to data imputation.

In one embodiment, the dataset may be trained to predict, based on any of the plurality of data from the subjects, a response to the treatment for a subject.

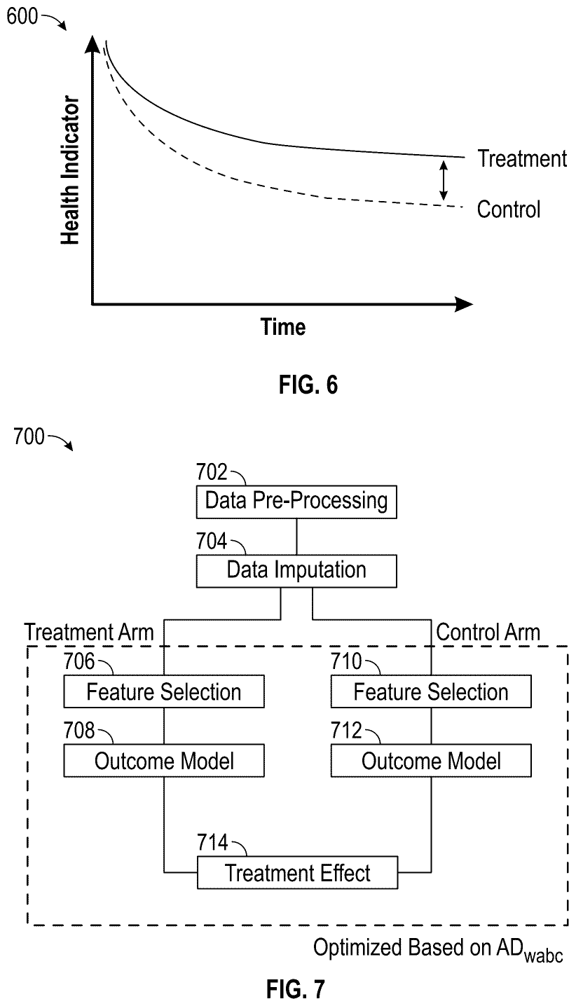

A summary of one embodiment of the method is illustrated in FIG. 7. As illustrated in FIG. 7, feature selection and outcome modeling are performed independently for the first arm, shown as the treatment arm, and the second arm, shown as the control arm. The treatment effect is determined by each of the treatment arm and the control arm and may be used to optimize the method.

FIG. 7 shows a schematic representation of the process for optimizing treatment effect predictions using multimodal data. FIG. 7 illustrates the workflow for both the treatment arm (706 and 708) and the control arm (710 and 712), highlighting the independent yet parallel processing paths for each group. The process begins with data imputation 702 and 704, which plays an important role in handling missing data points in the dataset. This step ensures that the subsequent analysis is based on a complete dataset, thereby enhancing the reliability of the predictions. Data imputation is applied to both the treatment arm 706 and control arm 710, ensuring consistency across the dataset. Following data imputation 702, feature selection 706 is performed for the treatment arm. This step involves identifying the most relevant features from the dataset that are likely to influence the outcome for subjects receiving the treatment. Similarly, feature selection 710 is conducted for the control arm, focusing on features that impact the outcome for subjects not receiving the treatment. The independent feature selection for each arm allows for tailored models that account for the specific characteristics of each group. Once the relevant features are selected, outcome models 708 and 712 are developed for the treatment arm and control arm, respectively. These models predict the health outcomes based on the selected features. The outcome model for the treatment arm predicts the expected results for subjects receiving the treatment, while the outcome model for the control arm predicts the outcomes for those not receiving the treatment. The outcome model for the treatment arm may then be used to predict the outcome for all subjects, whether they initially received the treatment or not, if they had received the treatment. Conversely, the outcome model for the control arm may be used to predict the outcome for all subjects, whether they initially received the treatment or not, if they had not received the treatment.

The final component, treatment effect 714, represents, for each subject, the difference between the predicted outcome if they had received the treatment and the predicted outcome if they had not received the treatment. This step is important as it quantifies the impact of the treatment by comparing the predicted outcomes, at a subject level, from both models 708 and 710. The optimization of the treatment effect 714 is based on maximizing the AD_{wabc} metric, which measures the models ability to accurately identify subjects who would benefit most from the treatment. This optimization process ensures that the model is fine-tuned to provide the most accurate and beneficial predictions for clinical decision-making.

In some embodiments, a nested cross-validation may be utilized. The nested cross-validation may comprise dividing the dataset into an outer training dataset and an outer testing dataset. In some embodiments, the outer testing dataset is further divided into inner training and inner testing datasets. In one embodiment, the cross-validation is used to generate the machine learning models. Of course, validation may be performed according to any type of validation contemplated by a person of ordinary skill in the art.

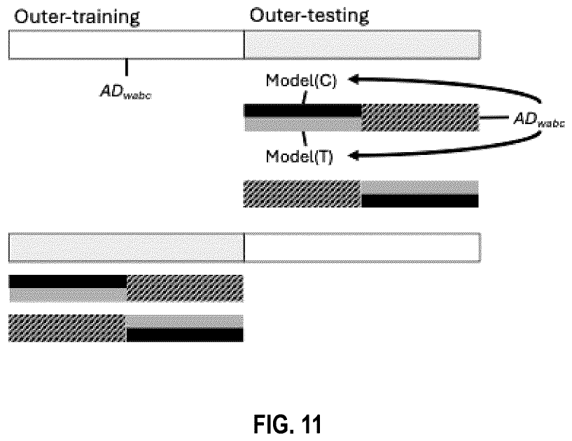

In one embodiment, the cross-validation process is employed to generate machine learning models by systematically partitioning the dataset into distinct subsets for training and testing. This approach involves dividing the dataset into an outer training dataset and an outer testing dataset, as illustrated in FIG. 11. The outer training dataset is further divided into an inner training dataset and an inner testing dataset, for example, through a k-fold cross-validation method. During each iteration of the cross-validation, the inner training dataset may be used to train the machine learning models, while the inner testing dataset is utilized to evaluate the model's performance. This iterative process allows for the optimization of model parameters and feature selection, ensuring that the models are robust and generalizable (e.g., widely applicable). The training engine 212, as depicted in FIG. 2, may play a crucial role in executing this process by leveraging the processing capabilities of the system 102.

The cross-validation process may be repeated multiple times, with different partitions of the dataset, to minimize overfitting and to provide a comprehensive assessment of the models predictive capabilities. By leveraging this technique, the machine learning models are refined and validated, ultimately leading to the selection of the best-performing model based on predefined metrics, such as accuracy, precision, recall, or the AD_{wabc} metric. This ensures that the models are well-suited for generating reliable clinical predictions from multimodal medical data, as facilitated by the inference engine 214 shown in FIG. 2. The predictive models 113, once trained, are deployed for inference, as depicted in FIG. 3, allowing for the generation of clinical predictions based on the multimodal medical data received by the system.

The present disclosure may contemplate a method for multimodal analysis of treatment outcome, wherein the predictive and/or statistical model is improved. As a nonlimiting example, the predictive and/or statistical model may be used to assess the risk associated with a particular treatment. This initial run employs the various contemplated data sources (multimodal) to quantify a risk factor. Subsequently, the determined risk value may be integrated as one of the analytical parameters (multimodal component) in subsequent predictive and/or statistical model runs. This incorporation enables a feedback loop mechanism wherein the model iteratively refines its predictions based on the calculated risk, thereby enhancing the accuracy and reliability of subsequent analyses. By integrating the assessed risk into the model's framework, in effect, it may continually adapt and evolve, improving its ability to forecast outcomes.

The aforementioned concept may offer several advantages. Firstly, by considering risk as a dynamic factor within the model, it may enhance the predictive and/or statistical model's predictive capabilities and/or accuracy by accounting for real-time data and feedback. Secondly, the iterative nature of a feedback element induces continuous improvement, ensuring that the model remains relevant in evolving applications. Moreover, this approach enables improvement to the model efficacy by utilizing the model itself, thus, allowing for the determined risk variable to be tailored to a given application or use case.

In an embodiment, the method may be configured to enhance the model through the consolidation of multiple input data points (e.g., each of the multimodal inputs) into a singular data point. As a nonlimiting example, a first set of distinct input factors may be collected, each representing specific parameters or characteristics relevant to the models analysis or the underlying trial. These multimodal factors may encompass diverse aspects such as biologics, radiomics, genetics, and the like. Subsequently, a computational process may be employed to amalgamate or combine these factors into a unified secondary factor. This consolidation may be achieved through algorithms or mathematical transformations that synthesize the underlying information, enabling the creation of a comprehensive and representative single factor. For the purposes of this description, a unified secondary factor may be a consolidated data point derived from multiple distinct input factors, synthesized to represent a comprehensive and holistic parameter for enhanced analysis within a predictive model.

Furthermore, the consolidated factor can be modified, weighted, generated, or otherwise tailored to better model outcomes. This customization process may allow for the optimization of the model's performance by emphasizing the importance of certain factor parameters or adjusting the influence of individual data points within a given factor based on the relative or absolute significance. Such tailoring may involve adding or fine-tuning weighting coefficients applied to each factor within the consolidated factor, thereby enhancing the model's sensitivity to critical factor aspects while mitigating the impact of less significant factor aspects. By enabling refinement and optimization of the consolidated factor, this approach may enhance the accuracy and efficacy of the model, facilitating more precise and reliable outcomes.

There may be different risk predictions based on each consolidated factor, group of features, or the like. In one embodiment, the different risk predictions may be combined and used for risk mitigation. However, in a further embodiment, the conditions that provides the greatest risk may be selected as the overall risk, and that resulting risk score may be integrated into the outcome calculation. Thus, the risk score may be solely based on the signal most likely to provide the greatest risk, which obviates the need to perform risk assessment for the remaining signals.

The method may contemplate simulation of treatment to determine risk level (e.g., whether there will be relapse or not). To perform such an analysis, models previously trained for the same or related treatment, trial, or cohort, from an earlier date (e.g., three years ago) may be utilized to determine the quality of progression and determine whether the treatment poses low, medium, or high risk.

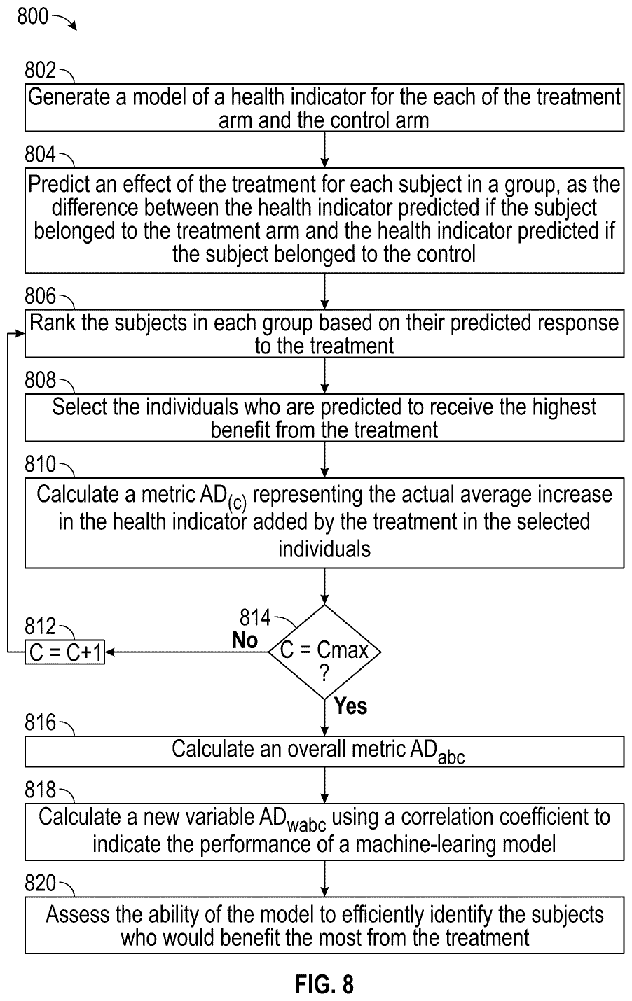

The flowchart shown in FIG. 8 demonstrates a method facilitating an improved multimodal pipeline for treatment outcome prediction as contemplated by the instant disclosure.

FIG. 8 illustrates a method 800 that supports an enhanced multimodal pipeline for predicting treatment outcomes, as facilitated by the system 102 depicted in FIG. 1A and FIG. 1B. This method 800 plays a significant role in assessing the effectiveness of the predictive models 113 in identifying subjects who would gain the most from a specific treatment.

The method 800 may begin with developing models predicting an health indicator of a cohort of individuals, independently for a cohort having received the treatment (treatment arm) and the cohort that did not received the treatment (control arm), in step 802. This step 802 may involve ingesting, reconciling, pre-processing, and aggregating multimodal data using the data management engine 210 shown in FIG. 2. In some embodiments, different multimodal variables are used for the treatment arm and control arm models. This step 802 may further involve training multimodal models using the training engine 212 shown in FIG. 2.

Next, in step 804, the method 800 may predict the health indicator based on multimodal data for each subject of a group if they had received the treatment and if they had not received the treatment. The health indicator predicted if they had received the treatment may be based on the model generated for the treatment arm and may be inferred by the inference engine 214 in FIG. 2. The health indicator predicted if they had not received the treatment may be based on the model generated for the control arm and may be inferred by the inference engine 214 in FIG. 2. The step 804 may further involve predicting an effect of the treatment for each subject in a group. In an embodiment, this prediction is calculated as the difference between the health indicator predicted if the subject belonged to the treatment arm and the health indicator predicted if the subject belonged to the control arm. In will be apparent to those skilled in the art that other metrics may be used, such as a ratio between the health indicators predicted with and without a treatment. This step may assist in quantifying the impact of the treatment on individual health outcomes, leveraging the processing capabilities of the system 102.

Following this, the subjects in each group may be ranked based on their predicted response to the treatment in step 806. In such an embodiment, this ranking allows for the identification of individuals who are likely to experience the most significant benefits from the treatment in step 808.

The method 800 may then select the individuals who are predicted to receive the most significant benefit from the treatment in step 808. This selection process focuses on optimizing treatment allocation to enhance positive health outcomes.

Subsequently, in an embodiment, a metric AD_{(c)} is calculated in step 810, representing the actual average increase in the health indicator added by the treatment in the top-ranked individuals compared to the average in the whole population, where c indicates the percentage of individuals that are included in the top-ranked group. Since a value of c equal to 1 means that all individuals are included in the top-ranked individuals, the value of AD_{c=1} is 0 by definition. For example, this metric provides a quantitative measure of the treatment's effectiveness for the chosen subjects. In some embodiments, the value c is initially set to an initial starting value, for example, 0.

The method 800 includes a loop where the variable c is incremented by a pre-determined increment in step 812. The increment may be by 0.1 or 10%, but it will be evident to those skilled in the art that other increments are possible. A check is performed in step 814 to determine if c has reached a maximum value cₘₐₓ.

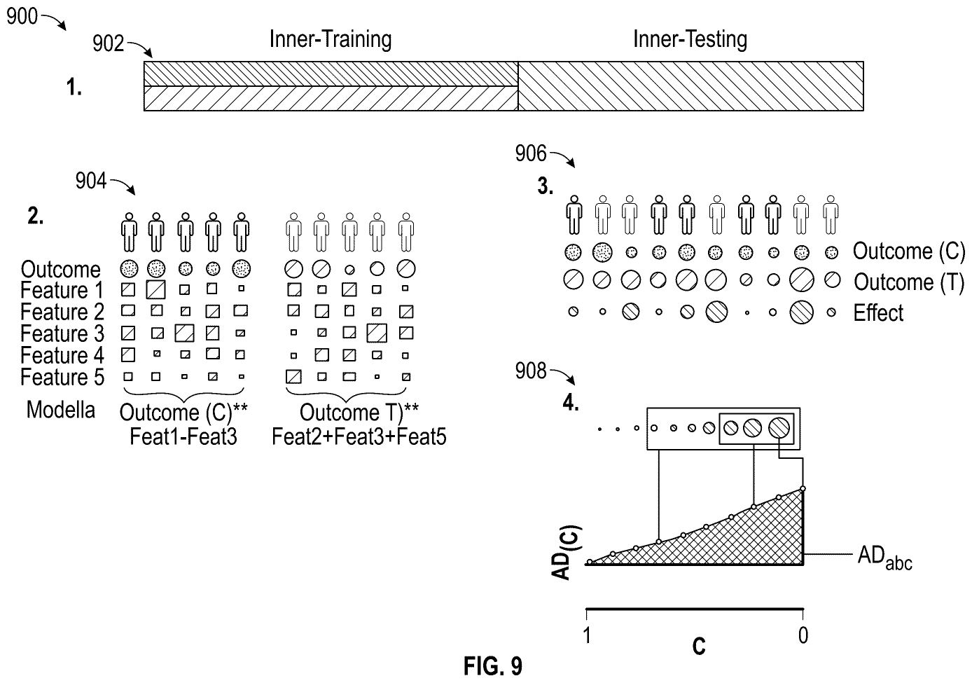

Once the loop condition is satisfied, an overall metric AD_{abc} may be calculated in step 816. In an embodiment, this metric is calculated as the integer of the AD_{(c)} values between cₘᵢₙ and cₘₐₓ, providing the area under the curve as illustrated in FIG. 9, based on the equation

$${AD}_{a b c} = {\int }_{c max}^{c min} ( {AD}_{( c )} ) d c .$$

A new variable AD_{wabc} may then be calculated in step 818 by integrating a correlation coefficient w between AD_{(c)} and c. The integration of a correlation coefficient w increases the score AD_{wabc} of scenarios where AD_{(c)} increases monotonously with c. The incorporation of w thereby serves to weigh down inferred scenarios where a small proportion of the population benefits disproportionately from the treatment. The variable AD_{wabc} may be calculated as AD_{wabc}=|ρ|*AD_{abc}, where ρ may be the Spearman correlation coefficient. It will be apparent to those skilled in the art that other types of transformation are possible.

Different models may be compared in step 820, and the model with the highest value of ADwabc may be selected by the training engine 212 depicted in FIG. 2.

In conclusion, in step 820 the method 800 evaluates the ability of the model to efficiently identify the subjects who would benefit the most from the treatment. This evaluation is important for validating the model's effectiveness in clinical decision-making and personalized treatment planning, as facilitated by the system architecture shown in FIG. 5.

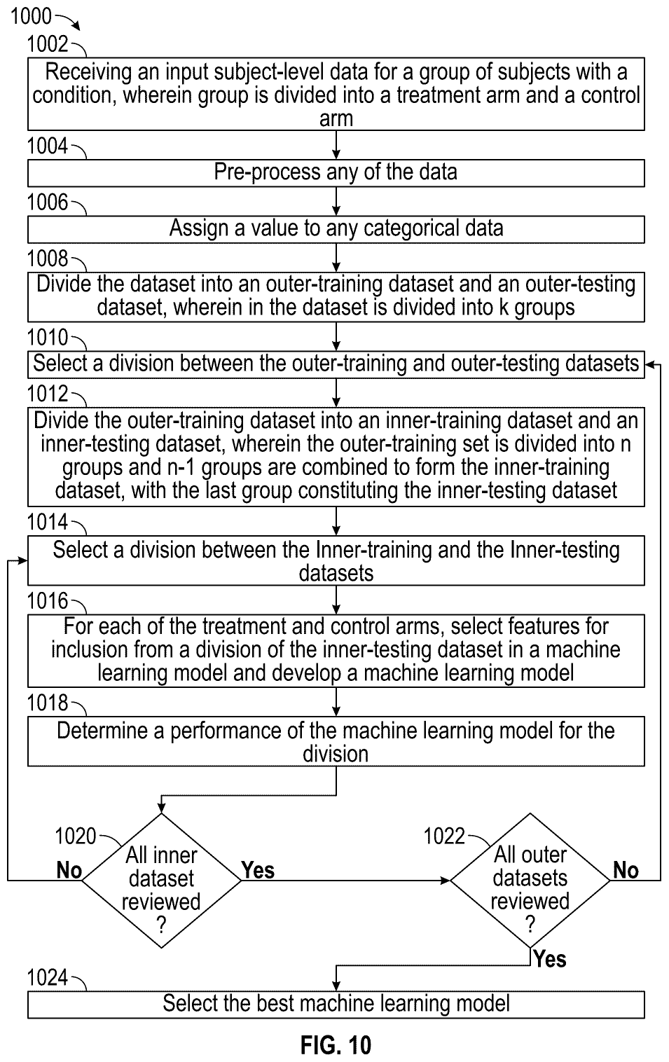

The flowchart shown in FIG. 10 demonstrates a method facilitating an improved multimodal pipeline for treatment outcome prediction as contemplated by the instant disclosure. One embodiment of the cross-validation is illustrated in FIG. 11.

FIG. 10 shows a flowchart diagram implementing a method 1000 for selecting an optimal machine learning model based on subject-level data for a group of subjects with a condition, as facilitated by the system 102 depicted in FIG. 1A and FIG. 1B. The process begins with receiving input subject-level data 1002, where the group is divided into a treatment arm and a control arm. This division is important for comparing the effects of treatment versus no treatment on the subjects.

The next step involves pre-processing any of the data in step 1004, which may be necessary for ensuring the data is clean and ready for analysis. This may include standardizing formats, removing errors, and normalizing values, as managed by the data management engine 210 shown in FIG. 2. Preprocessing the data may be beneficial in the practical application of reviewing and assessing multimodal health data from a patient to determine treatment efficacy or treatment outcomes because it ensures that the data is in a consistent and usable format for analysis. The data management engine 210, as shown in FIG. 2, handles various types of data, including genomic data (e.g., sequencing reads and genetic variant information), clinical data (e.g., patient demographics and medical history), radiological data (e.g., CT and MRI images), and biological data (e.g., blood test results and proteomic profiles). By standardizing formats, removing errors, and normalizing values, the engine prepares these diverse datasets for accurate and reliable input into predictive models. Pre-processing data may further involve automatically classifying images using trained machine learning and associating images with, for example, the tissue or tissues they show and the method used to obtain them. The data management engine 210 may further be used to automatically extract features from raw genomic data and images. This pre-processing step allows for integrating various data types into a cohesive dataset that can be effectively analyzed. In practical terms, this provides that healthcare providers can trust the outputs of the predictive models, as they are based on harmonized data, leading to more accurate assessments of treatment efficacy and better-informed clinical decisions for personalized patient care. Furthermore, the pre-processing step helps manage large and complex datasets, allowing statistical models to harness all existing information, independently of their starting format.

Following preprocessing, the method 1000 may include assigning and/or imputing a value to any variable with missing data (e.g., both numerical and categorical data types) in step 1006, which addresses missing or incomplete data points by assigning values based on statistical methods or assumptions. In assessing treatment outcomes, this process addresses gaps in patient information, such as missing genomic markers or incomplete clinical histories, to prevent biases in analysis. Practically, it allows for more accurate treatment efficacy assessments, as predictive models can utilize a full set of data points for informed predictions. Techniques like mean imputation or k-nearest neighbors ensure imputed values are representative, enhancing the reliability of treatment outcome predictions and enabling better-informed healthcare decisions.

The dataset is then divided into an outer-training dataset and an outer-testing dataset in step 1008, with the dataset being divided into n groups. A division between the outer-training and outer-testing datasets is selected in step 1010, ensuring that the model is trained and validated effectively.

In an embodiment, the outer-training dataset is further divided into an inner-training dataset and an inner-testing dataset in step 1012, where the outer-training set is divided into n groups and n−1 groups are combined to form the inner-training dataset, with the last group constituting the inner-testing dataset in step 1012. As a nonlimiting examples, this nested division facilitates a more granular approach to model training and validation, enhancing the model's accuracy and reliability, as supported by the training engine 212 depicted in FIG. 2.

In an embodiment, a division between the inner-training and inner-testing datasets is selected in step 1014, allowing for iterative refinement of the model. For example, for each of the treatment and control arms, features are selected for inclusion from a division of the inner-testing dataset in a machine learning model in step 1016, and a machine learning model is developed. This step is important for tailoring the model to the specific characteristics of the treatment and control groups, ensuring that the model captures the nuances of the data. In multimodal data analysis, tailoring the model to treatment and control groups involves selecting features from the inner-testing dataset, integrating diverse data types like genomic sequences, clinical histories, radiological images, and biological markers. This process ensures the model captures subtle differences influencing treatment outcomes. By reflecting each group's unique profiles through chosen features, the model can predict patient responses to treatments, considering genetic predispositions, medical history, imaging findings, and lab results. This results in more personalized predictions, enhancing treatment efficacy assessments and supporting targeted healthcare interventions.

The performance of the machine learning model for the division is determined in step 1018, which involves evaluating the model's predictive capabilities and accuracy. This assessment is important for identifying the most effective model configuration, leveraging the processing capabilities of the system 102. The performance of the machine learning model is of a benefit to the overall system of treatment outcome prediction because it influences the accuracy and reliability of predictions, enabling healthcare providers to make informed decisions based on precise data analysis. By assessing the model's performance, the system can identify the most effective configuration, optimizing computational resources to process complex multimodal data and deliver personalized treatment recommendations with high confidence.

The process may include a check in step 1020 verifying that all inner datasets have been used as the training/testing dataset following the established plan to ensure comprehensive evaluation and optimization. Once all inner datasets have been used as testing/training following the established plan, the process may include a condition to ensure all outer datasets have been successively used as the outer training dataset in step 1022. Once all outer datasets are reviewed, a machine learning model is selected in step 1024 based on the AD_{wabc} score, which measures the model's ability to accurately identify subjects who would benefit most from the treatment. The system 102 may use statistical analysis methods such as correlation coefficients and average treatment effect calculations to derive the AD_{wabc} score, which evaluates each model's ability to consistently and accurately identify subjects who would benefit most from treatment, thereby selecting the model that optimizes treatment efficacy predictions. This selection is significant as it ensures that the system benefits from having an optimal model to determine treatment efficacy and effect, as facilitated by the system architecture shown in FIG. 5.

As illustrated in FIG. 11, the outer-training dataset may be divided into an inner-training dataset and an inner-testing dataset. In one embodiment, the outer-training dataset is divided into n groups and n−1 groups are combined to form the inner-training dataset, with the last group constituting the inner-testing dataset. The inner-training dataset may comprise a plurality of data associated to subjects belonging to each of the first arm, illustrated in FIG. 11 as Model(T), and the second arm, Model(C), for inclusion in the model. The plurality of features may, in some embodiments, include the variable to assess the health of the subjects. In some embodiments, this health indicator is considered as the outcome and machine learning models specific to each arm are trained to predict it. It will be apparent to those trained in the art that other outcomes can be predicted by the models.

Further, in an embodiment, a first model is trained to predict the health indicator based on the selected plurality of features from the first arm and a second model is trained to predict the health indicator based on the selected plurality of features from the second arm. The first and second model may include at least one hyperparameter. In one embodiment, the at least one hyperparameter may be the same between the first and second model, however, in another embodiment, any of the at least one hyperparameters may vary between the first and second model. In some embodiments, the at least one hyperparameter may be determined according to the inner-testing dataset.

FIG. 9 illustrates one embodiment of the model generation process 900, described with reference to one outer-training dataset. In step 902 of FIG. 9, the outer-training dataset is divided into an inner-training and an inner-testing dataset, each comprising subjects from the first arm (shown above grey) and the second arm (shown above black). In step 904, a predicted outcome, which may be the predicted health indicator, is independently modeled based on a combination of the selected features for each of the first and second arms in the inner-training dataset.

Step 906 of FIG. 9 illustrates predicting an outcome of the subjects from the inner-testing dataset. The first and second model may be used to predict, for any of the subjects in the inner-testing group, the outcome if the subject had belonged to either the first arm or the second arm. In the embodiment illustrated in FIG. 9, the outcome is predicted in a first scenario where the subject is part of a control group Outcome(C) and a second scenario where the subject takes part in a treatment group Outcome(T). In one embodiment, the predicted effect of the treatment is determined according to the difference between the predicted Outcome(T) and Outcome(C). In an embodiment, the procedure may loop through the k divisions and the models may be constructed based on the inner-training dataset, while the prediction of treatment benefit may be based on the inner-testing group. For a given set of hyperparameters and selected variables, each model may be trained on the inner-training dataset and then may be evaluated on the corresponding inner-testing dataset. The process 900 may include repeating the procedure on the n folds/groups, and achieve n estimated AD_{wabc} metrics. Further, the process 900 may be configured to keep the subset of hyperparameters and selected variables that maximize the averaged AD_{wabc} metric over the n folds.

As illustrated in step 908 of FIG. 9, the process 900 may further comprise ranking each of the subjects based on the predicted effect of the treatment. In some embodiments, the process 900 may rank the subjects according to the subjects most likely to benefit from the treatment. In some such embodiments, a proportion c of the subjects may be selected. The proportion c of the subjects may be any of a percentage of the total number of subjects, the subjects having a likelihood of benefiting from the treatment over a threshold value, or any other deciding factor that may be desired. In one embodiment, a metric AD_{(c)} is calculated representing the actual average increase in the health indicator added by the treatment in the selected percentage c of subjects. In the embodiment illustrated in FIG. 9, the proportion c of the subjects is varied between 0 and 1 and, for each value of c, the an average benefit AD_{(c)} is computed for subjects representing a top c fraction. The process 900 may further comprise repeating the ranking of the subjects and ranking the subjects for various values c. The values of c may be any values that a person of ordinary skill in the art may desire, for example between 1 and 0.3.

An overall metric AD_{abc} is computed as the integer of the difference between AD_{(c)} and AD₍₁₎, for the varying values of c (area under the curve 908; FIG. 9):

$${AD}_{a b c} = {\int }_{c max}^{c min} ( {AD}_{( c )} ) d c .$$

The area under the curve above the value for c=1 (hashed area), which corresponds to the average treatment benefit, provides the metric AD_{abc}, which after incorporating the correlation coefficient between AD_{(c)} and c provides the performance of the model. In one embodiment, the Spearman correlation coefficient ρ between AD_{(c)} and c is calculated and a new variable AD_{wabc} is obtained as AD_{wabc}=|ρ|*AD_{abc}. The addition of ρ decreases the score for models providing a non-monotonous increase of AD_{(c)} with c, therefore penalizing models where the treatment benefit is maximal for a very limited number of subjects. The AD_{wabc} score is used to indicate the performance of the model. This metric assesses the ability of the model to efficiently identify the subjects who would benefit the most from the treatment.

However, in another embodiment, AD_{(c)} is computed as the ratio between the benefit of the c top-ranked fraction of the cohort and the average of the whole cohort. Further, in some embodiments, the correlation coefficient ρ may be omitted, so that AD_{abc} is directly used to optimize the models, which is contemplated to enable the maximization of the treatment effect for a subset of subjects without maximizing it for others.

In still another embodiment, the AD_{abc} may correspond to a mean, median, or product, or another summary statistic, of each AD_{(c)} values across the range of c values considered.

In still another embodiment, the metric used may correspond to the benefit gained for a given value of c or group of values of c. For instance, the model could be optimized based on the metric AD_{c=0.2} corresponding to the treatment benefit compared to the average for the top-ranked 20% individuals. It will be apparent to those skilled in the art that any value of c between 0 and 1 inclusive, might be used, with consequences on the characteristics of the optimal models.

In some embodiments, the system may utilize various methods to calculate and compare treatment effects. For instance, instead of using a difference to quantify the treatment effect, the system may employ a ratio or other comparative measures between the treatment and control outcomes.

The methods described in FIGS. 9 and 11 may be repeated a plurality of times with different divisions between inner-training and inner-testing. In one embodiment, the methods of FIGS. 9 and 11 are repeated n times, so that each of the n groups is consecutively the inner-testing dataset, with the n−1 other groups are used as the inner-training dataset. The repeat providing the highest value of AD_{wabc} is selected, effectively optimizing simultaneously, through an inner loop ensuring that all divisions are consecutively used as the inner-testing dataset, the feature selection for the first arm, the feature selection for the second arm, the hyperparameters for the first arm, and the hyperparameters for the second arm.

The method further comprises computing AD_{wabc} for the outer-testing dataset to determine the performance of the model.

Further, the methods described in FIGS. 9 and 11 may be repeated a plurality of times with different outer-training and outer-testing divisions. In one embodiment, the methods of FIGS. 9 and 11 are repeated k times. In one such embodiment, each of the k divisions may be consecutively used as an outer-testing group, with the k−1 other divisions used as the outer-training group. The model performance may, in some embodiments, be assessed on all the repeats, including the repeats nested within each outer-training division.

The method may determine the best model according to the repeat having the best AD_{wabc} score, and thus, the best performance.

The model can be used to identify the subcohort of patients who would benefit the most from the treatment. In some embodiments, the model can be used to evaluate the effect of a treatment, where the treatment specifically benefits a subcohort that is not known a priori. This will be apparent to those skilled in the art that such information can be valuable for stakeholders interested in drug development and validation.

The model can be used to predict the response of any individual subject according to their measured features. In some embodiments, the model can be incorporated in a data bank of predictive models 113 and be deployed for individual-level prediction for subjects that were not part of the original cohort. In some embodiments, the model can be used to predict the health indicator of the subject with treatment and without treatment.

In an embodiment, the system described is designed to help predict the outcomes of treatments over different periods of time, acting as a tool that can forecast how effective a treatment might be at various future points, such as 3 months or 1 year from now. Those skilled in the art will appreciate that it can be achieve for models specific to each treatment, by modelling the outcome at multiple time points. Temporal treatment benefits can also be obtained with models predicting the treatment benefit, either by optimizing the model on the treatment benefit at a given time point or by successively optimizing the model based on the treatment effect at each time point. The system may allow the user, such as a doctor or healthcare provider, to select a specific time frame for the prediction, enabling them to decide if they want to see how a treatment will work in the short term or in the long term. This flexibility is facilitated by the interface(s) 206, which allows users to input their desired prediction horizon.

By choosing different time horizons, the system can provide predictions that show how the effectiveness of a treatment might change over time. This is important because some treatments might work quickly, while others might take longer to show results. In such an embodiment, the data management engine 210 and the training engine 212 work together to adjust the predictive models based on the selected time frame, ensuring that the models are optimized for the specific temporal context.

As a nonlimiting example, if a treatment is predicted to be more effective after a year, a healthcare provider might decide to continue with the treatment for a longer period, or if the treatment shows quick results, the doctor might adjust the treatment sooner. The inference engine 214 then uses the refined models to generate accurate predictions, which are stored and managed within the system's database 112 and model bank 114. In essence, this feature helps tailor medical care to each patient's needs by providing insights into how treatments will perform over time, allowing for more informed decision-making.

### An Adaptable Multimodal Factory to Support Medical Decisions

While the aforementioned embodiments disclose the use of the method to predict treatment outcomes, it is contemplated that the disclosed method may be utilized for any purpose that may be contemplated or desired. For example, in some embodiments, the method can be applied to study non-diseased subjects subjected to different conditions. Further, in another embodiment, the method may be utilized with a group having a first condition and a group having a second condition. The conditions may, for example and without limitation, correspond to any of drugs taken, diet, living conditions, or other conditions that are contemplated.

In a further embodiment, the method may be utilized to assess the effect of the condition of any kind of indicator, for example, any of wealth, cognitive abilities, political opinion, or other indicators.

Still further, it is contemplated that in some embodiments, the method may be utilized to study non-medical systems. For example, the method may be utilized to study the effect of fertilizers on crops, effect of drugs on farm animals, or any other purpose that may be desired.

In an embodiment, the system is designed to operate both dependently and independently of a patient's natural history, providing a comprehensive framework for predicting health outcomes using multimodal data. When functioning independently, the system may be configured to simulate scenarios where no medical intervention is applied, effectively predicting the natural progression of a patient's condition. This capability provides a benefit for understanding the baseline trajectory of a disease, allowing providers to assess the potential risks and outcomes if no treatment is administered. By leveraging historical data and patterns inherent in the patient's health records, including genomic, clinical, radiological, and biological data, the system can generate predictions that reflect the natural course of the illness, offering valuable insights into the expected progression without medical intervention.

Conversely, when operating dependently on natural history, the system integrates this baseline information with potential treatment scenarios to evaluate the impact of various interventions using multimodal data. By comparing the predicted outcomes with and without treatment, the system can provide a nuanced analysis of the treatment's efficacy, highlighting the benefits and potential improvements over the natural course of the disease. This dual capability ensures that healthcare providers have a robust tool for decision-making, enabling them to weigh the advantages of intervention against the natural progression of the condition. The integration of multimodal data enhances this process by providing a rich, detailed view of the patient's health status, allowing for more accurate and personalized treatment planning. Ultimately, this approach optimizes care and improves patient outcomes by offering a clear picture of how different strategies may alter the patient's health trajectory.

The system is engineered to enhance diagnostic precision by leveraging early detection techniques, such as liquid biopsy, which allows for the identification of circulating tumor DNA or other biomarkers in the blood. This minimally invasive method provides crucial insights into the presence and progression of diseases like cancer at an earlier stage. By integrating liquid biopsy results with additional data points, such as the subject's age, imaging data, and clinical history, the system refines its diagnostic capabilities. In effect, this integration may improve the insights as typically derived from liquid biopsy and may improve the predictive accuracy of the multimodal pipeline described herein. This integration is achieved through the multimodal data pipeline, which efficiently combines and processes diverse data types to construct a comprehensive patient profile. The pipeline ensures that each data point, whether genomic, radiological, or clinical, is analyzed in conjunction with others, offering a holistic view of the patient's health status and enabling more accurate and timely diagnoses.

Incorporating liquid biopsy results into the multimodal data pipeline significantly enhances the system's ability to deliver personalized and precise diagnoses. By coupling early detection data with age-related factors and imaging results (or other data points), the system can uncover patterns and correlations that might be overlooked when data is analyzed in isolation. This comprehensive approach empowers healthcare providers to make more informed decisions, allowing them to tailor treatment plans to the specific needs and conditions of each patient. The multimodal pipeline's capacity to synthesize and analyze vast amounts of data ensures that the system remains adaptive and responsive to new information, continually refining its diagnostic capabilities. Ultimately, this integration of early detection methods with multimodal data not only improves diagnostic accuracy but also supports proactive healthcare strategies, potentially leading to better patient outcomes and more effective disease management.

In an embodiment, the system conducts preprocessing to effectively merge and aggregate diverse data types, ensuring that each dataset is prepared for comprehensive analysis. This aggregation process involves the preprocessing of each data type, which is crucial for integrating multimodal data such as genomic, clinical, radiological, and biological information. The data management engine 210, as depicted in FIG. 2, plays a pivotal role in this process by applying specific preprocessing methodologies tailored to the project's requirements or the algorithm's needs. For instance, when handling Variant Call Format (VCF) data, the system employs different preprocessing methods based on the subject's disease, ensuring that the genomic data is accurately represented and ready for analysis. When handling Variant Call Format (VCF) data, the system utilizes the data management engine 210 to apply disease-specific preprocessing methods, ensuring accurate representation of genomic data. This involves steps such as filtering out irrelevant variants, normalizing data formats, and aligning genomic information with clinical and radiological datasets, as outlined in the method 300. The processor 202 executes these preprocessing tasks, leveraging the memory 204 to store intermediate data and results, while the interface 206 facilitates communication between different system components. By tailoring preprocessing to the specific disease context, the system ensures that the training engine 212 can effectively use the refined data to develop robust predictive models, enhancing the accuracy of clinical predictions generated by the inference engine 214. This tailored approach allows the system to optimize preprocessing not only for the data type but also for the underlying condition or circumstance associated with each dataset.

The preprocessing methodologies are designed to be flexible and adaptive, accommodating the unique characteristics of each data type and the specific requirements of the analysis. For example, when assessing Variant Calling data for a patient with breast cancer, the system applies a specialized set of preprocessing considerations that account for the genetic markers and mutations relevant to the disease. The training engine 212, as shown in FIG. 2, utilizes these preprocessed datasets to train predictive models, ensuring that the models are robust and capable of delivering accurate clinical predictions. By optimizing preprocessing for both the data type and the condition, the system enhances its ability to generate reliable insights into treatment efficacy and patient outcomes.

In an embodiment, the inference engine 214, depicted in FIG. 2, leverages the preprocessed and aggregated data to generate clinical predictions that are informed by a comprehensive understanding of the patient's health status. This process ensures that healthcare providers have access to accurate and actionable information, allowing them to tailor treatment plans to the specific needs of each patient. By continuously refining preprocessing methodologies based on project and algorithm dependencies, the system remains adaptive and responsive to new challenges, ultimately improving patient care and advancing the field of personalized medicine.

In an embodiment, the inference engine 214, as depicted in FIG. 2, utilizes preprocessed and aggregated data to generate clinical predictions by applying computational algorithms that integrate both data type-specific and condition-specific preprocessing considerations. For example, when processing VCF data, the system uses specialized algorithms to filter and normalize genetic variants while incorporating disease-specific parameters, such as those relevant to breast cancer, to ensure the genomic data is accurately contextualized within the patient's health profile. This dual-layered preprocessing approach enables the system to produce precise and actionable clinical insights, allowing healthcare providers to customize treatment plans based on a comprehensive understanding of each patient's unique medical condition. By continuously adapting preprocessing methodologies to align with evolving project requirements and algorithmic advancements, the system enhances its predictive capabilities, thereby advancing personalized medicine and improving patient care outcomes.

The system described herein may be configured to seamlessly integrate with electronic medical records (EMRs) by injecting the results of treatment predictions and other algorithmic outputs directly into these records. This integration is facilitated by the interface(s) 206, as depicted in FIG. 2, which enables communication between the system and various EMR platforms through an Application Programming Interface (API). The API allows for the secure and efficient transmission of important predictive metrics, such as treatment prediction efficacy, to different medical record and hospital systems. By leveraging the processing capabilities of the processor(s) 202 and the memory 204, the system may ensure that healthcare providers have immediate access to the most current and relevant predictive data, enabling them to make informed decisions regarding patient care. The system's ability to interface with multiple EMR platforms underscores its versatility and adaptability in diverse healthcare environments, ensuring seamless integration and data exchange across various systems.

In technical terms, the integration of treatment prediction results into electronic medical records (EMRs) may involve the creation of dedicated sections within the EMR document that display key predictive metrics, such as treatment success rates or efficacy scores. These sections can be dynamically updated through the system's API, ensuring that healthcare providers have access to the latest predictive insights. The interface(s) 206, as depicted in FIG. 2, facilitate this integration by enabling seamless communication between the system and EMR platforms, allowing for real-time data updates. Additionally, these predictive metrics can serve as valuable inputs for EMR administrators, who may utilize them to generate further metrics related to patient outcomes, such as risk assessments or personalized treatment plans. By leveraging the processing capabilities of the processor(s) 202 and the memory 204, the system ensures that these metrics are accurately calculated and efficiently transmitted, enhancing the EMR's functionality and supporting more informed clinical decision-making.

FIG. 12 illustrates a sophisticated user interface tailored for clinicians who are conducting treatment efficacy assessments, for example, for kidney cancer patients. This interface may be an integral part of the multimodal factory system 102 described in FIG. 1A and FIG. 2, designed to streamline the data input process for busy healthcare professionals. The scroll-down menu on the left, allowing selection of a specific prediction model, may correspond to the model selection process described in the model bank 114 of FIG. 1A and FIG. 2. This feature may enable clinicians to choose models appropriate for different patient subgroups or specific clinical scenarios, enhancing the precision of their assessments.

The interactive computerized form on the right side of the interface in FIG. 12 collects a comprehensive set of patient data. The fields for tumor diameter (1), pathological stage (2), pathological node status (3), anesthesia score (4), diagnosis status (5), and surgical indication (6) represent the diverse multimodal data types. This detailed data collection supports the predictive model inference process described in step 310 of FIG. 3, allowing clinicians to input the nuanced patient information necessary for generating accurate clinical predictions.

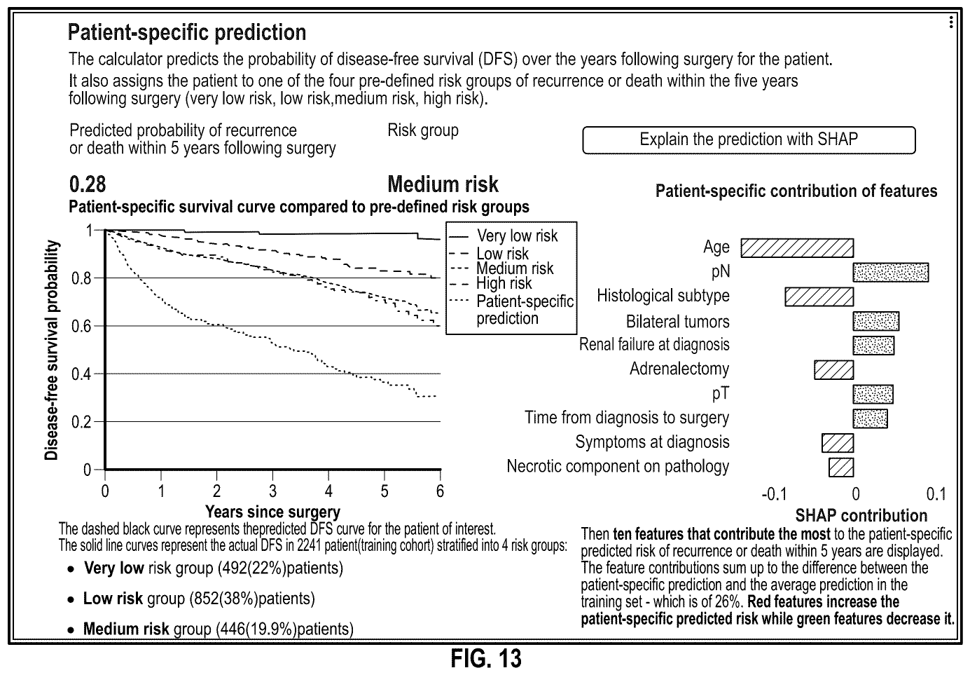

FIG. 13 presents a clinician-focused user interface displaying the results of a patient-specific prediction for kidney cancer. This interface may represent the output of the inference engine 214 described in FIG. 2, translated into a visually intuitive format for clinical interpretation. The graph on the left, showing disease-free survival probability over time, with the patient-specific prediction as a black dashed line, may be a direct implementation of the treatment effect module 714 output described in FIG. 6 and FIG. 7. This visual representation may allow clinicians to quickly gauge a patient's predicted outcome relative to various risk groups, facilitating informed discussions with patients about their prognosis and treatment options.

The right side of the FIG. 13 interface, displaying patient-specific contributions of various clinical features, aligns with the feature selection processes described in the treatment feature selection module 706 and control feature selection module 710 of FIG. 6 and FIG. 7. For clinicians conducting treatment efficacy assessments, this visualization may provide valuable insights into the factors driving the prediction for each patient. It may help implement the ranking of subjects based on predicted response to treatment, as outlined in step 806 of FIG. 8, allowing clinicians to identify patients who are likely to benefit most from specific interventions.

This comprehensive display may support the aforementioned assessment, enabling clinicians to efficiently evaluate the model's ability to identify subjects who would benefit most from the treatment. The clear presentation of both predicted outcomes and influencing factors may aid in clinical decision-making and in explaining the rationale behind treatment recommendations to patients.

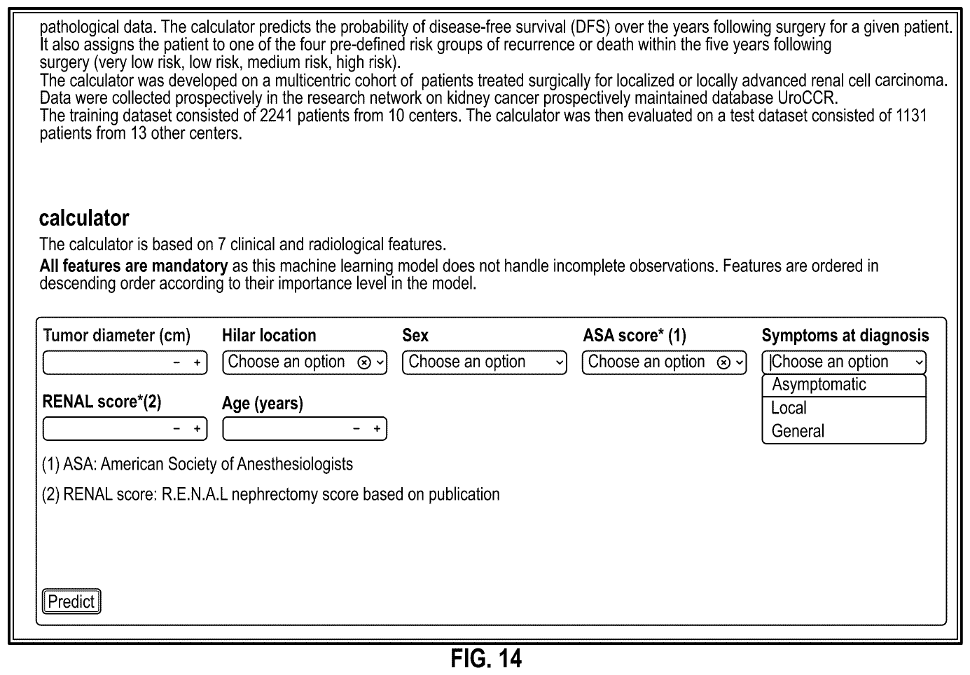

FIG. 14 depicts a prediction interface specifically designed for clinicians to assess the risk of pT3a upstage after nephrectomy. This interface may be an implementation of the computing device 106 described in FIG. 1A and FIG. 3, providing a user-friendly portal for clinicians to interact with the multimodal factory system 102. For clinicians conducting treatment efficacy assessments, the parameters such as tumor diameter, hilar location, sex, ASA score, and symptoms at diagnosis may be used, as illustrated in FIG. 14. This may allow for a direct comparison of predicted outcomes with and without intervention, supporting evidence-based decision-making in patient care.

The “Predict” button may initiate the process described in step 404 of FIG. 4, where clinical predictions are generated based on the entered multimodal medical data using corresponding predictive models. This feature may allow clinicians to quickly obtain patient-specific predictions during consultations, facilitating real-time clinical decision-making.

In the context of treatment effect determination, these interfaces play a vital role in facilitating the input of data, where health indicator models are generated and treatment effects are predicted for each subject. The standardized data entry through dropdown menus and editable fields may support the consistent application of the AD_{wabc} metric described in FIG. 9, enabling accurate comparison between predicted outcomes for treatment.

By providing a streamlined method for inputting patient data and generating predictions specifically for pT3a upstage after nephrectomy, this interface implements the patient-specific approach to treatment effect prediction described throughout the application. It may enable clinicians to efficiently apply the methods outlined in FIG. 8 and FIG. 10 in their daily practice, potentially improving the accuracy and consistency of treatment planning for kidney cancer patients.

As contemplated throughout the instant disclosure, the system may utilize various approaches for evaluating feature importance in predictive modeling, which may include, but are not limited to, cohort-level analysis and individual-level predictions. For cohort modeling, the contribution of each feature may be assessed by methods such as shuffling the values of the feature among individuals, one feature at a time, and calculating the effect on the model accuracy. This approach may provide insights into how different features impact the overall predictive performance of the model across the cohort. The system may, in some cases, iterate through features, reassigning their values among subjects using various randomization techniques, and measuring the resulting changes in model accuracy. Features that, when altered, lead to changes in model accuracy may be considered as potentially important for the prediction task.

For individual-level predictions, the system may employ alternative methods to evaluate feature importance. When processing a single subject's data using a trained model from the model bank, the system may, in some instances, analyze the impact of feature value variations on the prediction. This approach may offer insights into how changes in specific features could influence the prediction for a particular individual. The system may adjust the values of features using various methods, which could include systematic variations within predefined ranges or other modification techniques, and may observe and record the corresponding changes in the predicted outcome. Such methods may allow for a more tailored assessment of feature importance, potentially highlighting factors that could be relevant for an individual patient's prognosis or treatment response. These approaches may be adapted or combined in various ways depending on the specific requirements of the prediction task and the nature of the available data.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the claims.

What is claimed is:

1. A method for training predictive models to generate clinical predictions from multimodal medical data, comprising:
receiving, by a processor, multimodal medical data of one or more medical subjects;
preprocessing and aggregating, by the processor, one or more features of the multimodal medical data;
training, by the processor, one or more predictive models to generate a clinical prediction based on the one or more features of the multimodal medical data; and
deploying, by the processor, the one or more predictive models to support subject-level predictions.

2. The method of claim 1, wherein the multimodal medical data comprises any one or a combination of: genomic data, clinical data, radiological data, and biological data, and wherein the clinical prediction includes a prediction of the survival time.

3. The method of claim 1, wherein the clinical prediction includes a prediction of the progression-free survival time, wherein the clinical prediction includes a prediction of the occurrence of adverse events, and wherein the clinical prediction includes a prediction of the onset of a disease.

4. The method of claim 1, wherein for preprocessing, by the processor, the one or more features of the multimodal medical data, the method comprises any or a combination of:
when the multimodal medical data is received in the form of a plurality of unimodal medical data, reconciling the plurality of unimodal medical data to obtain the multimodal medical data of the one or more medical subjects;
imputing missing values in the multimodal medical data;
cleaning the multimodal medical data for errors; and
performing feature extraction on the multimodal medical data.

5. The method of claim 1, further comprising determining, by the processor, the contribution of each feature from the multimodal medical data to the clinical prediction, by:
for each feature associated with the multimodal medical data:
randomly shuffling values associated with the feature of medical subjects in the cohort of medical subjects to generate at least one pseudo-replicate dataset;
testing performance of the one or more predictive models on the pseudo-replicate dataset; and
estimating the feature contribution as the change in performance of the one or more predictive models between the multimodal medical data and the pseudo-replicate dataset,
wherein the contributions of each feature are reported to the user in a computer interface, and wherein the contributions of each feature are reported to the user in a downloadable report.

6. The method of claim 1, wherein the performance of the one or more predictive models is tested using nested cross-validation, wherein the one or more medical subjects are grouped into one or more cohorts of medical subjects based on at least one feature in the multimodal medical data, and wherein the one or more predictive models are trained for predicting the benefit of a given treatment option.

7. The method of claim 6, wherein the one or more predictive models are trained for identifying the subset of medical subjects most likely to benefit from a given treatment option.

8. The method of claim 6, wherein the one or more predictive models are trained for generating the clinical prediction for each treatment option associated with one or more diseases.

9. A system for generating clinical predictions from multimodal medical data, comprising:
a. a processor configured to ingest data belonging to different modalities and available in various formats;
b. a data management engine configured to reconcile the different types of data, creating a multilevel multimodal database wherein each datapoint, independent of the modality, is associated with a specific subject;
c. a feature extraction module configured to process the data and extract features of interest;
d. a data aggregation module configured to aggregate data from one or more modalities, produce a list of features associated with subjects, and impute missing data to obtain values or distributions of values for all features in each subject;
e. a model development engine configured to develop clinical prediction models based on one or more groups of subjects, and to optimize and assess model performance using a validation technique;
f. a model bank for storing trained models, containing trained models adapted to make predictions for new subjects based on user goals and inputting subject features; and
g. an interface for reporting individual-level predictions and feature contributions.

10. The system of claim 9, wherein the data comprises distinct files per subject or multiple data types within the same file, wherein the data includes imaging data, genomic data, clinical data, and biological data, wherein the imaging data comprises one or more of X-rays, MRI, PET scans, and CT scans, wherein the genomic data comprise one or more of sequencing reads, genetic variants, gene expression profiles, genomic profiles, and methylation profiles, wherein the clinical data comprises one or more of age, history, and health indicators, wherein the clinical data is collected in time series from a given starting point, and wherein the biological data comprises one or more of metabolomics, proteomics, pathology data, and results from blood or urine analyses.

11. The system of claim 9, wherein the feature extraction module utilizes tools to process images by automatically segmenting and extracting features comprising one or more of shape, intensity, and texture.

12. The system of claim 9, wherein the feature extraction module utilizes tools to process sequencing data to, one or more of, identify genetic variants, assess genomic profiles, establish gene expression patterns, and extract other genomic features.

13. A computer-implemented method to predict the effect of a treatment to a condition, comprising:
a. receiving multimodal data for at least two cohorts of subjects having received different treatments;
b. developing a model for a clinical outcome independently for each of the at least two cohorts of subjects;
c. calculating a treatment benefit based on the clinical outcomes predicted with each of the models developed for the at least two cohorts of subjects; and
d. optimizing the models based on the predicted treatment benefit.

14. The computer-implemented method of claim 13, wherein the multimodal data is selected from a group consisting of clinical data, biological data, genomic data, and radiomic data.

15. The computer-method of claim 13, wherein the received data is pre-processed prior to training the models, wherein the pre-processing comprises one or more of any of quality checks and data cleaning, data imputation, data normalization, image processing, and analyses of genomic data.

16. The computer-implemented method of claim 13, wherein different features are selected for the models for each of the at least two different cohorts of subjects, wherein the feature selection is integral to the step of optimizing the models.

17. The computer-implemented method of claim 13, the step of calculating the treatment benefit further comprising comparing the clinical outcome predicted for each subject based on the model developed on the cohort having received a treatment and the clinical outcome predicted for each subject based on the model developed on the cohort not having received the treatment.

18. The computer-implemented method of claim 17, wherein the comparison comprises computing the difference between the clinical outcome predicted with the two models, wherein the comparison further comprises the steps of:
a. defining a proportion c of individuals benefitting the most of the treatment; and
b. calculating the treatment benefit AD_{(c)} as the added average benefit observed in the top-ranked fraction c of the individuals compared to the average of the cohort.

19. The computer-implemented method of claim 18, wherein the comparison further comprises the steps of:
a. calculating AD_{(c)} for varying values of c; and
b. calculating the treatment benefit AD_{abc} as the integer of AD_{(c)} across the range of c values tested.

20. The computer-implemented method of claim 19, wherein the comparison further comprises the steps of:
a. calculating the correlation coefficient ρ between AD_{(c)} and c; and
b. calculating the treatment benefit AD_{wabc} by multiplying AD_{abc} by the absolute value of ρ.
